(19) United States
(12) Patent Application Publication
(10) Pub. No.: US 2012/0029321 A1
(43) Pub. Date: Feb. 2, 2012

(54) AIRWAY ADJUNCT RESUSCITATION SYSTEMS AND METHODS

(75) Inventors: Michael Makaretz, Yarmouth, ME (US); Keith Lurie, Minneapolis, MN (US); Kurt Krueger, Stacy, MN (US); Greg Voss, Lakeville, MN (US); Anja Metzger, Stillwater, MN (US)

(73) Assignee: Advanced Circulatory Systems, Inc., Roseville, MN (US)

(21) Appl. No.: 13/189,330

(22) Filed: Jul. 22, 2011

Related U.S. Application Data
(60) Provisional application No. 61/368,150, filed on Jul. 27, 2010.

(51) Int. Cl. A61B 5/00; A61H 31/02

(52) U.S. Cl. 600/301; 601/43

(57) ABSTRACT

Embodiments of the present invention encompass systems and methods for administering intrathoracic pressure and cooling treatments to patients suffering from or at risk of developing heart failure, cardiac arrest, sepsis, shock, acute respiratory distress syndrome, polytrauma, head disease, elevated hepatic or portal vein pressures, bleeding during abdominal, head and neck surgery, or insufficient circulation during open heart surgery.

### CROSS-REFERENCES TO RELATED APPLICATIONS

This application is a nonprovisional of, and claims the benefit of the filing date of, U.S. Provisional Patent Application No. 61/368,150 filed Jul. 27, 2010 (Attorney Docket No. 80118-788149), the entire content of which is incorporated herein by reference for all purposes. This application is also related to U.S. patent application Ser. No. 12/119,374 filed May 12, 2008 (Attorney Docket No. 016354-006400US), the content of which is incorporated herein by reference for all purposes.

### BACKGROUND OF THE INVENTION

Embodiments of the present invention relate generally to the field of cardiopulmonary resuscitation and, in particular, to techniques to increase circulation when performing cardiopulmonary resuscitation (“CPR”).

Despite current methods of CPR most people die after cardiac arrest. One of the major reasons is that blood flow to the heart and brain is very poor with traditional manual closed chest CPR. Greater circulation of blood during CPR would result in improved outcomes.

CPR has traditionally been performed by repetitively compressing the chest and intermittently providing positive pressure ventilation. Each time the chest is compressed and then allowed to recoil, blood circulates to the heart and brain; and each time a breath is delivered, the lungs fill with oxygen. This approach is extremely inefficient, in part, because each positive pressure ventilation results in an increase in pressure within the thorax and a consequent reduction in venous blood flow back to the heart. In addition, each positive pressure breath increases intracranial pressure and thereby reduces cerebral blood flow.

Multiple methods may be used when performing CPR in patients in cardiac arrest. In this life-threatening situation, the heart is not capable of circulating blood, so non-invasive external means are used to assist in the circulation of blood to the vital organs, including the heart, lungs, and brain. The methods and devices that may be used to circulate blood during cardiac arrest usually include the manipulation of one or more of a patient's body parts, usually the chest, to increase the magnitude and duration of the patient's negative intrathoracic pressure. The most common methods include manual closed chest CPR, active compression/decompression (ACD) CPR, mechanical CPR with manual or automated devices that compress the chest and either allow the chest to recoil passively or actively, and devices that compress the chest wall and then function like an iron lung and actively expand the thoracic cage. Some of these approaches and devices only compress the anterior aspect of the chest, such as the sternum, while other approaches and devices compress all or part of the thorax circumferentially. Some approaches and devices also compress the thorax and abdomen in an alternating sequence. Some approaches also involve compressing the lower extremities to enhance venous blood flow back to the heart and augment arterial pressure, so that more blood goes to the brain. Other approaches also involve compressing the back while the patient is lying on his/her stomach. Some devices include the non-invasive methods and devices outlined above that are coupled with invasive devices, such as an intra-aortic balloon, and devices to simultaneously cool the patient.

Because the cardiac valves remain essentially intact during CPR, blood is pushed out of the heart into the aorta during the chest compression phase of CPR. When the chest wall recoils, blood from extrathoracic compartments (e.g., the abdomen, upper limbs, and head) enters the thorax, specifically the heart and lungs. During the chest wall recoil phase, blood fills the cardiac chambers as well as the coronary arteries, i.e., the arteries that provide blood to the heart muscle. Without the next chest compression, the blood would pool in the heart and lungs during cardiac arrest, as there is insufficient intrinsic cardiac pump activity to promote forward blood flow. Thus, chest compressions are an essential part of CPR.

Blood flows to the brain during both the chest compression and decompression phases. The amount of blood flow to the brain depends upon the gradient between forward blood flow (determined in large part by the arterial pressure) and the resistance in flow into the brain (determined in large part by the intracranial pressure).

During the compression phase of closed chest manual (standard) CPR, air is pushed out of the thorax and into the atmosphere via the trachea and airways. During the decompression phase, air passively returns back into the thorax via the same airway system. As such, respiratory gases move out of and back into the thorax. With each compression the pressure within the chest is nearly instantaneously transmitted to the heart, and also to the brain via the spinal column and vascular connections. Thus, with each external chest compression, pressure is increased in the thorax and within all of the organs in the thorax.

A variety of impeding or preventing mechanisms may be used to prevent or impede respiratory gases from flowing back into the lungs, including those described in U.S. Pat. Nos. 5,551,420; 5,692,498; 6,062,219; 5,730,122; 6,155,257; 6,234,916; 6,224,562; 6,986,349; and 7,204,251, the complete disclosures of which are herein incorporated by reference. The mechanisms may be configured to completely prevent or provide resistance to the inflow of respiratory gases into the patient while the patient inspires. In devices that completely prevent the flow of respiratory gases, the valves may be configured as pressure responsive valves that open after a threshold negative intrathoracic pressure has been reached. Such systems and devices are referred to herein collectively by the name “impedance threshold device” or “ITD”. Other examples of such ITDs are described in U.S. Pat. Nos. 6,526,973 and 6,604,523, incorporated herein by reference. However, it will be appreciated that a wide variety of devices may be used. As another example, devices may be interfaced with a person's airway to prevent respiratory gas flow to the person's lungs during a portion of an inhalation event to enhance circulation and decrease intracranial pressure, including those described in U.S. Pat. No. 7,195,012, incorporated herein by reference.

Methods and devices, such as ITDs that reduce the amount of respiratory gases inside the thorax by preventing said gases from reentering the thorax during the chest wall recoil phase, or by actively removing said gases either intermittently or continuously, result in less and less air in the thorax. Less air in the thorax makes room for more and more blood to return to the heart during the chest wall recoil phase. Application of the aforesaid methods and devices cause a reduction in intrathoracic pressures, either during the chest wall recoil phase or continuously during the chest compression and decompression phases, which results in a simultaneous decrease in intracranial pressures. As such, application of these methods and devices increases circulation to the coronary arteries during the chest wall decompression phase, and increases blood flow to the brain during the compression and decompression phases, thereby delivering more oxygen-rich blood to the brain.

Known techniques have failed to take a systems-based approach that includes methods and devices that are optimized to interface with the patient's airway, provide the benefits of ITD therapy and maximize circulation to the heart and brain by compressing and decompressing the chest. Such an approach would be desirable since it may result in an overall increase in the likelihood of a positive outcome after cardiac arrest.

As previously mentioned, traditional or standard CPR also includes the delivery of a positive pressure breath periodically, in order to inflate the lungs and provide oxygen (“O₂”). In addition, positive pressure ventilation provides a means to remove carbon dioxide (“CO₂”) from the lungs. Since the delivery of O₂ is an important aspect of CPR, periodic positive pressure ventilation traditionally needs to be delivered to inflate the lungs and provide oxygen. However, recently some harmful effects of positive pressure ventilation have been demonstrated. See, K. Lurie et al.; “Hyperventilation-induced hypotension during cardiopulmonary resuscitation,” Circulation; 2004 Apr. 27; 109(16):1960-5, incorporated herein by reference. Each time positive pressure ventilation is delivered, intrathoracic pressure rises. The rise in intrathoracic pressure results in an immediate reduction in venous blood flow back to the heart, and an immediate rise in intracranial pressures, thereby resulting in greater resistance to forward blood flow to the brain. This occurs when the chest compressions are delivered continuously or with periodic pauses for a positive pressure breath. When chest compressions are stopped in order to deliver a positive pressure breath (which is currently recommended by the American Heart Association when the airway is not secured by a ventilation tube such as an endotracheal tube), blood flow to the heart and brain nearly ceases. Without the chest compressions to serve as a pump during the period of time a positive pressure breath is delivered, there is no circulation of blood to the heart and brain.

With traditional CPR, the lungs need to be regularly inflated to provide O₂ to the lungs and to support movement of blood through the pulmonary vasculature. O₂ exchange is inadequate without positive pressure ventilation, especially for prolonged resuscitation efforts, and the lungs develop atelectasis or collapse, making blood flow through the lungs more difficult as the pulmonary vascular resistance becomes too high. See K. Lurie et al.; “Comparison of 10 versus 2 breaths per minute strategy during cardiopulmonary resuscitation in a porcine model of cardiac arrest,” Journal of Respiratory Care; 2008, in press, incorporated herein by reference. Thus, periodic inflation of the lungs provides O₂, helps to clear CO₂, and helps to reduce pulmonary vascular resistance (resistance to blood flow through the lungs) by preventing lung collapse.

However, in light of these recently discovered advances in understanding the physiology of blood flow, and the effects of positive pressure ventilation on blood flow to the heart and brain, as well the resistance to blood flow through the lungs, new methods and devices are needed that: a) obviate the need for positive pressure ventilation, b) provide a means to lower intrathoracic pressure during CPR (to augment venous blood flow back to the heart and lower intracranial pressures), and c) still provide a means to prevent lung collapse. Accordingly, the present invention provides new methods, systems and devices that optimize circulation and respiration during CPR while avoiding the harmful effects of positive pressure ventilation.

### BRIEF SUMMARY OF THE INVENTION

In one embodiment, the invention provides a method for performing cardiopulmonary resuscitation which comprises interfacing with a person's airway an airway system that includes at least a first lumen and a second lumen. CPR chest compressions may be repeatedly performed on the person, and simultaneously with the chest compressions, a continuous vacuum may be applied to the airway. In one embodiment, the continuous vacuum may be applied to the first lumen of the airway system. In one embodiment, the continuous vacuum may be applied for a period of time ranging from 10 seconds to the end of the CPR chest compressions. Simultaneously, an effective amount of O₂ gas may be injected into the person's lungs through the second lumen at a high velocity. By applying continuous vacuum to the patient's airway and simultaneously insufflating O₂ into the lungs at a high velocity sufficient to circulate O₂ into the alveoli, the present invention provides significantly greater blood flow to the heart and brain during CPR, and thereby provides an improved method for resuscitation without the necessity of positive pressure ventilation.

In one embodiment, the continuous vacuum applied to the first lumen may be about −2 mmHg to about −20 mmHg. In another embodiment, the velocity of the O₂-rich gas may be about 20 ft./sec. to about 1100 ft./sec. In still another embodiment, additional steps may be added wherein the continuous vacuum may be discontinued and positive or negative pressure ventilation may be supplied through the first lumen to the patient with or without the CPR chest compressions and with or without the injection of high velocity oxygen gas through the second lumen.

In one embodiment, negative intrathoracic pressure may be maintained at least in part by using an impedance threshold device that prevents respiratory gases from returning to the patient's thorax during the decompression phase of each CPR chest compression. In another embodiment, the CPR chest compressions may be performed using closed chest CPR, active compression/decompression CPR, or mechanical CPR with a manual or automated device that compresses the chest wall and either allows the chest to recoil passively or actively re-expands the thoracic cage of the patient. In another embodiment, the delivery of O₂ gas and/or the application of continuous vacuum may be regulated based upon one or more physiological measurements such as airway pressure, intracranial pressure, O₂ saturation, end tidal CO₂, transcutaneous lactate, pH measurements, and the like.

In another embodiment, the invention provides a cardiopulmonary resuscitation system for use during the performance of CPR chest compressions on a patient. The CPR resuscitation system may comprise an airway system configured to interface with a patient's airway. The airway system includes at least a first and a second lumen, with the first lumen being configured to ventilate the patient's lungs during the CPR chest compressions. A source of oxygen gas may be coupled to the second lumen, which may be configured to inject an effective volume of oxygen gas from the source of oxygen gas into the patient's lungs at high velocity during the CPR chest compression. Means may also be provided for applying a continuous vacuum to the person's airway simultaneously with the injection of oxygen gas and the performance of CPR chest compressions, at least for a period of time ranging from 10 seconds to the end of the CPR chest compressions. In one embodiment, the continuous vacuum is applied for at least 15 seconds and in some cases for at least 30 seconds. For example, the vacuum means may comprise a source of continuous vacuum coupled with the first lumen. The airway system may further comprise at least a second lumen configured to be coupled with a source of O₂, so that O₂ gas may be injected at high velocity into the person's airway through the second lumen during the performance of the repeated CPR chest compressions and the application of continuous vacuum through the first lumen.

In one embodiment, the first lumen of the airway system may comprise the central lumen of a ventilation tube, e.g., an endotracheal tube or a supraglottic airway adjunct, and the second lumen may comprise one or more small diameter (e.g., about 0.025-1.0 cm) tubules or cannula positioned within the ventilator tube's central lumen. In another embodiment, the airway system may further comprise an impedance threshold device configured to prevent respiratory gases from flowing into the person's airway. In other embodiments, the resuscitation system may include a valve system configured to discontinue the application of continuous vacuum and thereafter supply positive pressure ventilation to the person' airway through the first lumen of the airway system. Such valve systems may include a fish mouth valve that is closed when continuous vacuum is being applied to the first lumen and is opened when positive pressure ventilation is applied to the first lumen. Another example of such a valve system may comprise a piston and a pair of rolling diaphragms that are movable between a first position that allows the application of continuous vacuum to the first lumen and seals off the source of positive pressure ventilation to the first lumen, and a second position that allows the application of positive pressure ventilation to the first lumen and seals off the source of continuous vacuum to the first lumen.

In one embodiment, the continuous vacuum may be regulated by one or more regulators configured to generate a negative airway pressure of between about −2 mmHg and about −20 mmHg, and, in another embodiment, a pressure gauge may be incorporated to measure airway pressures and/or intrathoracic pressure during application of the resuscitation system. In another embodiment, the resuscitation system may include a controller comprising control valves connected to a microcontroller to regulate the application of continuous vacuum and the delivery of high velocity O₂ gas in one phase, and the application of positive airway pressure to the person in a second phase.

In another embodiment, the invention provides a locking supraglottic airway system comprising an airway tube having a central lumen with a proximal supraglottal section and a distal esophageal section. The system may further comprise means for applying a continuous vacuum to the central lumen; means for advancing the airway tube into a patient's airway; a first inflatable cuff positioned in the esophageal section of the airway tube and configured to seal off the esophageal area of the patient's airway when inflated; and a second inflatable cuff positioned in the supraglottal section of the airway tube and configured to seal off the laryngeal area of the patient's airway when inflated. The second cuff may comprise an extension configured to seal off the nasopharyngeal area of the patient's airway when inflated. The first and second cuffs act to maintain a negative intrathoracic pressure in the patient's airway when a vacuum is applied to the central lumen.

In one embodiment, the locking supraglottic airway system may further comprise one or more tubules disposed in the central lumen and configured to deliver oxygen to ventilation ports in the second cuff, thereby injecting oxygen at high velocity into the patient's airway. In another embodiment, the means for advancing the airway tube into the patient's airway may comprise a pilot tube running the length of the exterior of the airway tube and an orogastric tube. The pilot tube may be configured to slide over and be guided by the orogastric tube after the orogastric tube has been positioned in the patient's airway.

For a fuller understanding of the nature and advantages of the present invention, reference should be had to the ensuing detailed description taken in conjunction with the accompanying drawings.

### BRIEF DESCRIPTION OF THE DRAWINGS

FIG. 1 is a hemodynamic tracing from a pig study wherein the pig receives CPR in accordance with embodiments of the present invention;

FIG. 2 is a cross-sectional view of one embodiment of a CPR resuscitation system in accordance with embodiments of the present invention;

FIG. 3 is a perspective view of another embodiment of a CPR resuscitation system device of the invention;

FIG. 4 is a cross-sectional view of another embodiment of a CPR resuscitation system in accordance with embodiments of the present invention having a fish mouth valve mechanism;

FIG. 5a is cross-sectional view of a valve device in accordance with embodiments of the present invention showing the position of a rolling piston mechanism during positive pressure ventilation of a patient;

FIG. 5b is a cross-sectional view of the valve device of FIG. 5a showing the position of the valve mechanism during the application of continuous vacuum to the patient;

FIG. 6a is a cross-sectional view of another valve device according to embodiments of the present invention showing the closed position of the valve mechanism;

FIG. 6b is a cross-sectional view of the valve device of FIG. 6a showing the open position of the valve mechanism;

FIG. 6c is a cross-sectional view of a resuscitation system according to embodiments of the present invention employing the valve device of FIG. 6a/b;

FIG. 7a illustrates one perspective view of a Locking Supraglottic Airway in accordance with embodiments of the present invention;

FIG. 7b illustrates another perspective view of the Locking Supraglottic Airway of FIG. 7a;

FIG. 7c is a sagittal view of patient's airway interfaced with the Locking Supraglottic Airway of FIG. 7a/b;

FIG. 8 is a block diagram showing the components of an automated control system in accordance with embodiments of the present invention.

FIGS. 9A and 9B illustrate aspects of an airway adjunct resuscitation system according to embodiments of the present invention.

FIG. 10 illustrates aspects of an airway adjunct resuscitation system according to embodiments of the present invention.

FIG. 11 illustrates aspects of an airway adjunct resuscitation system according to embodiments of the present invention.

### DETAILED DESCRIPTION OF THE INVENTION

In one embodiment, the invention provides a method for performing cardiopulmonary resuscitation which comprises: 1) interfacing an airway system with a patient's airway, wherein the airway system includes at least a first lumen and a second lumen; 2) repeatedly performing CPR chest compressions on the patient; and simultaneously with the CPR chest compressions 3) applying a continuous vacuum to the first lumen for a period of time ranging from 10 seconds to the end of the CPR chest compressions; and 4) injecting an effective volume of oxygen gas into the person's lungs at high velocity through the second lumen.

As used herein, including the appended claims, the “patient” may include any subject undergoing cardiopulmonary respiration (CPR), and may include both human and non-human animals.

As used herein including the appended claims, the phrase “airway system” is intended to include any system that is adapted to be interfaced with a patient's airway and has at least one lumen adapted to ventilate the patient's lungs during CPR; i.e. is adapted to move respiratory gases into and out of the patient's lungs. Such airway systems are sometimes referred to herein as “airway adjuncts” or “ventilation tubes”. Non-limiting examples of airway systems may include endotracheal tubes, supraglottic airway devices, Combitubes, obturator airways, laryngeal mask airways, and the like. Airway systems of the present invention also comprise at least a second lumen adapted to deliver oxygen gas into the patient's lungs.

As used herein including the appended claims, the phrase “CPR chest compressions” is intended to include any of the aforementioned CPR methods having a chest compression phase and a chest decompression (or recoil) phase. The chest compression phase serves to increase intrathoracic pressure and, thus, generate a pressure gradient between the thorax and the rest of the body, which in turn forces blood to the brain and other extra-thoracic organs. In addition, the chest compression phase causes the collapse of some of the bronchioles and, as a result, gas that is trapped in the distal portions of the airways is compressed. Thus, when there are respiratory gases in the lungs, the chest compression phase can help to open up the lungs and thus prevent atelectasis (collapse of the lungs). CPR chest compressions may also help to adequately exchange respiratory gases and help to maintain blood flow, as long as the lungs are partially inflated during the chest decompression phase. As a result, tissue oxygenation is maintained at a high level, CO₂ can be removed, and blood can move from the right heart to the left heart with a better match between perfusion and ventilation. In the context of the present invention, CPR chest compressions may be viewed as providing a motor, and the combination of continuous high velocity O₂-rich gas delivery and the application of a continuous vacuum to the patient's airway may be viewed as optimizing or improving the blood circulation to the heart and brain that is produced by that motor. In addition, the present invention may optimize the delivery of O₂ to and CO₂ removal from the patient's lungs.

The CPR chest compressions may also generate decompression phase negative intrathoracic pressure with each chest wall recoil. An ITD may be used to prevent respiratory gases from returning to the thorax during the chest wall recoil of the decompression phase of each CPR chest compression. By preventing respiratory gases from reentering the lungs during the decompression phase of CPR, the ITD helps maintain the decompression phase negative intrathoracic pressure. However, even when an ITD is used, the level of decompression phase negative intrathoracic pressure during standard CPR may oscillate with each compression and decompression cycle. This oscillation may result in a failure to maintain a continual negative intrathoracic pressure since at the peak of the oscillation, intrathoracic pressure may reach values that are at or above atmospheric pressure.

As used herein, the phrase “continuous vacuum” means that, when simultaneously combined with CPR chest compressions and the injection of high velocity O₂ in accordance with the invention, the application of vacuum to the patient's airway is not interrupted for a period of time ranging from 10 seconds to the end of the CPR chest compressions. In some cases it could be for at least 15 seconds and in other cases at least 30 seconds to the end of performing CPR. In one embodiment of the invention, a continuous vacuum is applied to the patient's airway at a level sufficient to supplement the decompression phase negative intrathoracic pressure in the patient and remove respiratory gases from the patient's airway. In some embodiments, the continuous vacuum may be applied to the patient's airway by connecting a vacuum source to the lumen of an airway system such as an endotracheal tube. In other embodiments, the continuous vacuum may be applied to the patient's airway by other means; e.g. through a connector for the vacuum source at a remote location in a ventilation circuit or through a separate lumen, such as a nasal tube. As described above, the values of the intrathoracic pressure provided by the continuous vacuum may oscillate; e.g. with each CPR chest compression, and therefore the intrathoracic pressures values may not remain continuously negative relative to atmospheric pressure. However, it is understood that the negative pressure (vacuum) applied to the patient's airway will remain continuously negative for at least 10 seconds during the performance of CPR chest compressions and the injection of high velocity O₂.

The oxygen gas injected into the patient's lungs in accordance with the invention is sometimes simply referred to herein, including the appended claims, as “O₂”. It is understood that the term “O₂” is intended to include mixtures of oxygen and other gases. In some embodiments, the second lumen through which O₂ is delivered may be incorporated within the first lumen. For example, the first lumen may comprise the central lumen of a ventilation tube, e.g., an endotracheal tube, through which the second lumen may be disposed, and a continuous vacuum may be applied and maintained in the central lumen of the tube, e.g. through a valve mechanism or impedance threshold device.

In one embodiment of the invention, the volume of O₂ delivered via the second lumen is sufficient to result in adequate oxygenation of the alveoli of the lungs (sometimes referred to herein, including the appended claims, as an “effective volume” or an “effective O₂ volume”). In one embodiment, an effective O₂ volume may be in the range of about 1 liter to about 20 liters delivered to the lungs during one minute of CPR chest compressions. Accordingly, these effective O₂ volumes may be referred to herein in units of “liters per minute” or “L/min”. In some embodiments, an effective O₂ volume of between about 3 L/min and 15 L/min may be preferred. In other embodiments, an effective volume may be about 12 L/min. In one embodiment, the second lumen is positioned within the patient's airway so as to deliver an effective O₂ volume in close proximity to the patient's carina tracheae.

The velocity at which the effective O₂ volume is injected into the lungs in accordance with the invention is largely dependent on the diameter of the delivery lumen. In one embodiment, an effective O₂ volume may be delivered through one or more tubules having a lumen diameter small enough to generate what is sometimes referred to herein, including the appended claims, as a “high velocity” flow of O₂ or “high velocity O₂”. As used herein, including the appended claims, the term “high velocity” is intended to mean a velocity that is high enough to inject an effective O₂ volume into the patient's lungs without interfering with the generation and maintenance of a continuous vacuum in the patient's airway. In one embodiment, high velocity O₂ may have a velocity in the range of about 20 ft/sec to about 1100 ft/sec. In order to generate high velocity O₂, the diameter of the lumen delivering the effective O₂ volume may be in the range of about 0.1 cm to about 1.0 cm in some embodiment. In other embodiments, the lumen diameter may be about 0.25 cm to about 1.0 cm.

The injection of high velocity O₂ into the patient's lungs through the trachea may produce a laminar or turbulent flow pattern. The flow pattern will depend upon a number of factors including the volumetric flow rate, O₂ velocity, size of the one or more tubules used to inject the high velocity O₂, and the size and architectural characteristics of the receiving airway system. Optimizing the degree of laminar and/or turbulent flow patterns may help to improve the overall efficiency of the invention. For example, in one embodiment O₂ may be delivered as a high velocity O₂ laminar flow in one direction primarily in the middle of the trachea, bronchi, and bronchioles. As a result, the flow of gases in the reverse direction resulting from the applied vacuum may move closer to the walls of these structures. Accordingly, a simultaneous bidirectional exchange of respiratory gases can occur in a relatively efficient manner. Physiological feedback sensors that measure flow and pressure, for example, may provide a means to further optimize the flow characteristics and, thus, the efficiency of the invention. Other physiological sensors may provide a similar kind of benefit.

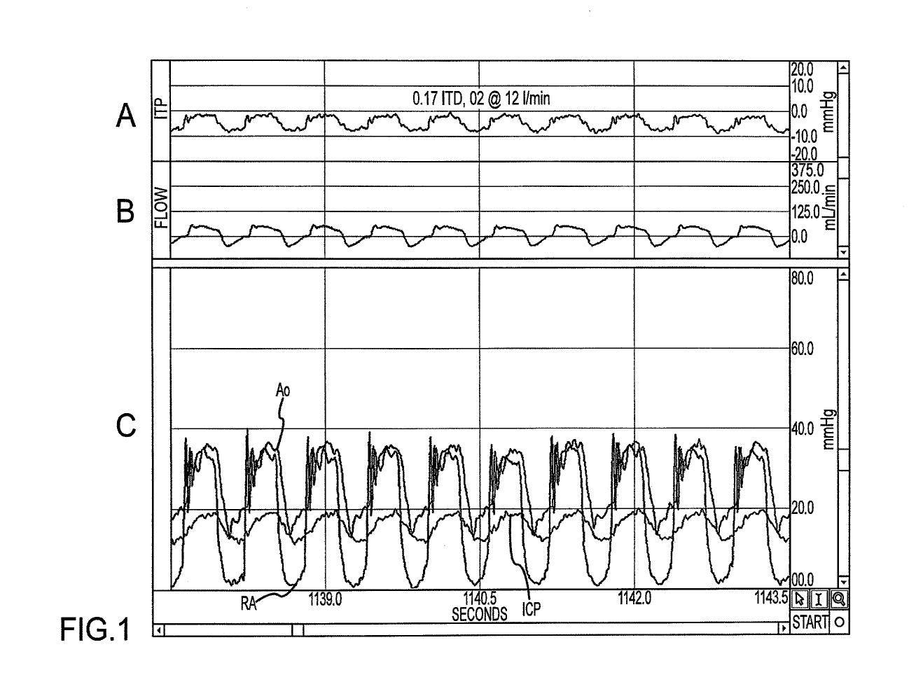

FIG. 1 shows several hemodynamic tracings that illustrate the results of a pig study wherein CPR was performed in accordance with one embodiment of the invention. A 30 kg pig was placed into ventricular fibrillation with methods previously described in: “Hyperventilation-induced hypotension during cardiopulmonary resuscitation,” Circulation; 2004 Apr. 27; 109(16):1960-5, incorporated herein by reference. After 8 minutes of untreated cardiac arrest, CPR compressions were performed at 100 times per minute using an automated CPR device at a depth of 25% of the anterior-posterior diameter of the pig. After each compression, the automated CPR device pulled the compressing pad upwards to allow for the natural recoil of the chest wall in an unimpeded manner. During the time the automated CPR device was activated, a continuous vacuum was pulled via an endotracheal tube, and an ITD with a cracking pressure of 0.17 lbs, was used to provide a resistance of −9 mmHg. About 12 L/min of 100% oxygen was delivered through a single 1 mm (0.1 cm) diameter tube inserted into the lumen of the endotracheal tube to provide high velocity O₂ of about 830 ft/sec.

Tracing panel A in FIG. 1 depicts the intrathoracic pressure (“ITP”) in mmHg as measured in the trachea of the pig by a micromannometer-tipped catheter. It can be seen from tracing panel A that a continual negative ITP was maintained during the entire 9 minutes of CPR and oscillated with each compression and decompression cycle between −1 and −9 mmHg. Tracing panel B of FIG. 1 depicts blood flow to the carotid artery in mL/min as measured with a Doppler flow probe around the carotid artery, and shows how blood flow may vary with each compression and decompression cycle. Tracing panel C of FIG. 1 depicts the changes in aortic pressure (Ao), right atrial pressure (RA) and intracranial pressure (ICP) during CPR in accordance with the invention, and shows how the values for Ao, RA and ICP increase and decrease with each compression and decompression cycle. The difference between the values for Ao and RA in the decompression phase is called the “coronary perfusion pressure,” and the difference between the values for Ao and ICP is called the “cerebral perfusion pressure.” With the present invention, lung oxygenation is maintained at clinically acceptable values, and coronary and cerebral perfusion pressure is maintained at a level adequate to allow for the return of spontaneous circulation after a cardiac arrest. The arterial and venous blood gases, after 9 minutes of CPR in accordance with the invention, were as follows: arterial blood pH=7.26, pCO₂=48, pO₂=396, HCO₃=22, base excess=−5, and % saturation=100%; and the venous blood pH=7.17, pCO₂=74.6, pO₂=20, HCO₃=27, base excess=−1, and % saturation=21%.

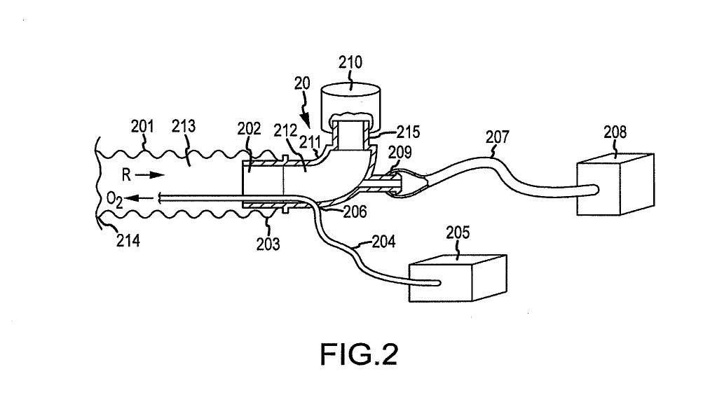

A device 20 suitable for the practice of one embodiment of the invention is shown in FIG. 2. Device 20 may comprise a housing 211 that defines a central lumen 212. A ventilation tube 201 comprising central lumen 213 may be connected to the patient's respiratory system at its distal end 214 and may be attached to device 20 at fitting 202, which communicates with central lumen 212. As used throughout the description provided herein, the term “ventilation tube” refers to any airway system having a central lumen through which respiratory gases may pass, e.g., an endotracheal tube, laryngeal mask airway device, supraglottic airway device, etc. High velocity O₂ may be delivered into proximal end 203 of ventilation tube 201 through one or more tubules (cannulae) 204 that extend from O₂ source 205 into central lumen 212 of device 20 through opening 206. Tubule(s) 204 may run the length of ventilation tube 201 and direct a flow of high velocity O₂ into the patient's respiratory system at the distal tip 214 thereof, as shown by the arrow labeled “O₂”. In one embodiment, the high velocity O₂ may be delivered at a velocity of between 20 and 1100 ft/sec and the diameter of tubules 204 may be between 0.025-1.0 cm, depending upon the number of tubules 204 used.

A vacuum line 207 connected to a vacuum source 208 may be attached to device 20 at fitting 209, which communicates with central lumen 212 of device 20. When activated, vacuum source 208 generates a continuous vacuum in lumen 212 of device 20 and lumen 213 of ventilation tube 201, which results in a negative intrathoracic pressure in the patient's airway and lungs. This vacuum may generate a flow of respiratory gases R from the patient's respiratory system into lumen 213 of ventilation tube 201 and lumen 212 of device 20. An impedance threshold device (ITD) 210 may be attached to device 20 at fitting 215, which communicates with lumen 212. ITD 210 may be any of the known ITDs that prevent or impede respiratory gases R from flowing back into the patient's respiratory system thereby helping maintain negative intrathoracic pressure. Examples of ITDs may be found in the aforementioned U.S. patents previously incorporated herein by reference. ITD 210 may be set to maintain a negative intrathoracic pressure between about −2 mmHg and about −20 mmHg, and preferably between about −6 mmHg and about −12 mmHg. Optionally, one or more gauges to assess changes in pressure within device 20 could be attached, for example, via a Y-connector attached to fitting 209, 211, or another connection to device 20. Such gauge(s) may be used to provide the user with information regarding the pressure within device 20 at any point in time.

Device 20 may be activated by turning on O₂ source 205 and vacuum source 208 as soon as ventilation tube 201 is inserted into the patient's airway. In some cases, O₂ source 205 may be turned on before vacuum source 208. Simultaneously with the injection of O₂ and the application of continuous vacuum, CPR chest compressions on the patient may be performed until there is a successful resuscitation, or other CPR procedures are performed. The continuous vacuum may be regulated by ITD 210, which opens at the preset cracking pressure, such that the intrathoracic pressure in the patient's respiratory system remains below atmospheric pressure, e.g. never exceeds a predetermined negative intrathoracic pressure value. Further, if the patient starts to breath during CPR or after a successful resuscitation the inspiratory resistance may never be greater that that to which ITD 210 is set. Thus, ITD 210 not only serves to regulate the applied vacuum but also provides a safety feature so that the patient can breathe, if spontaneous respiratory efforts are present during the CPR effort. Once the patient has been resuscitated and CPR is no longer performed, vacuum line 207 may be disconnected, or vacuum source 208 may be switched off if connected to a switch.

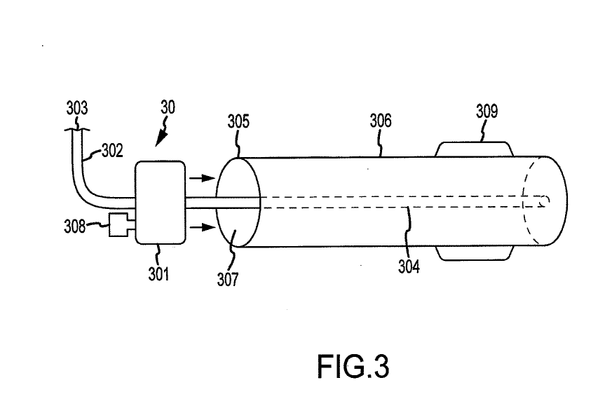

In another embodiment, the means for delivering high velocity O₂ may be incorporated into the central lumen of a standard ventilator tube and may be separate from the means for applying the continuous vacuum. For example, in the embodiment illustrated in FIG. 3, adaptor 30 may comprise a body 301 having an attached O₂ cannula 302 extending therethrough. O₂ cannula 302 may comprise a proximal end 303 attached to an O₂ source (not shown) and a distal portion 306. Body 301 may be adapted to be coupled with proximal end 305 of standard endotracheal tube 306 with the distal portion 304 of tubule 302 positioned within the central lumen 307 of endotracheal tube 306 so that tubule 302 extends substantially the entire length of endotracheal tube 306 and directs high velocity O₂ into the patient's respiratory system. This arrangement enables personnel administering CPR to deliver high velocity O₂ into the distal portions of a standard endotracheal tube 306, and simultaneously pull a continuous vacuum in lumen 307 using another separate device. Adapter 30 may also include one or more optional sideline attachments 308 to measure airway pressures, temperature, O₂ saturation, end tital carbon dioxide (ETCO₂), transcutaneous lactate, pH and a variety of other physiological parameters and/or respiratory metabolites Endotracheal tube 306 may also contain standard attachments, e.g., endotracheal cuff 309.

In another embodiment, the present invention may be used in combination with traditional CPR methods that employ the periodic delivery of positive pressure ventilation to the patient's respiratory system; e.g. to expand the lungs fully. This additional step may in some cases add further benefit, particularly in a setting of prolonged resuscitations. Although such positive pressure ventilation is optional in the practice of the invention, it may serve a function which is the equivalent of a sigh during normal respiration in a healthy person. Both the sigh and intermittent positive pressure ventilation help to recruit more alveoli in the lungs, which may help prevent collapse and/or closure of the smaller airways and some alveoli.

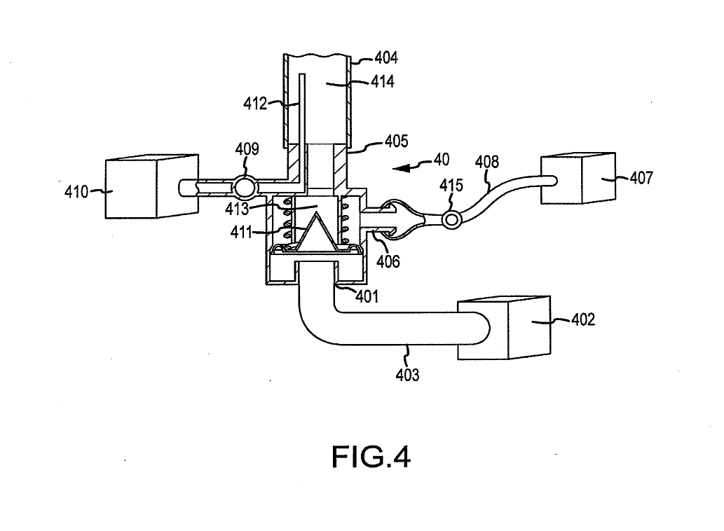

Accordingly, in some embodiments of the invention, the patient may be ventilated actively during traditional CPR with either positive or negative pressure ventilation before or after the performance of CPR in accordance with the invention. For example, in one embodiment of the invention, a valve device may be attached to a source of positive pressure ventilation, such as a resuscitator bag, a mechanical ventilator or an anesthesia machine, so that ventilation may be applied immediately before or after CPR in accordance with the invention without having to change equipment. As one non-limiting example, device 40 shown in FIG. 4 may be connected at port 401 to mechanical ventilator 402 through ventilator circuit 403. Device 40 may also be connected at fitting 405 to the patient's airway through ventilation tube 404 having central lumen 414. Device 40 may also be connected at fitting 406 to vacuum source 407 through vacuum line 408 and switching mechanism 415, and connected through switching mechanism 409 to O₂ source 410. The supply of O₂ may be turned off and on using switching mechanism 409, so that high velocity O₂ may be used during CPR according to the invention and then either used or not used during traditional CPR.

Regulator valve 411 in FIG. 4 may serve as a vacuum regulator during the practice of the invention, and may also facilitate positive pressure ventilation via the same circuit if the patient needs positive pressure ventilation before or after the performance of CPR in accordance with the invention. Valve 411 may be a diaphragm with an integrated valve that is capable of opening at a predetermined pressure differential that is greater than the differential required to move the diaphragm, e.g., a “fish mouth” or “duck bill” valve. When used in accordance with the invention, valve 411 may preferably be fixed at one vacuum level, e.g. −8 to −9 mmHg, but the vacuum level may be varied with an alteration in the fish mouth valve design.

When clinically indicated, positive pressure ventilation may be periodically delivered to the patient through ventilator circuit 403 by opening and closing valve 411. The delivery of high velocity O₂ to the patient may be provided through tubule 412 into lumen 414 and may be switched off and on using switch 409. A vacuum may be applied through vacuum line 408 to provide a continuous vacuum at a predetermined level in central lumen 413 of device 40 and ventilation tube lumen 414 by switching on switch 415 and closing valve 411. Alternatively, the supply of continuous vacuum may be switched off by switch 415 and valve 411 may be opened to provide for periodic positive pressure ventilation when necessary or desirable.

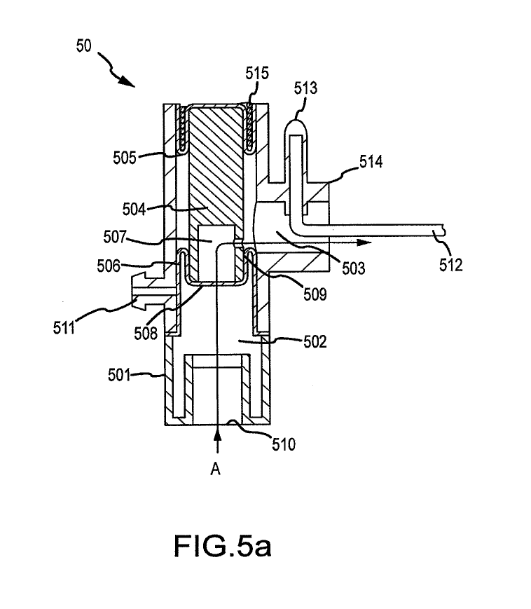

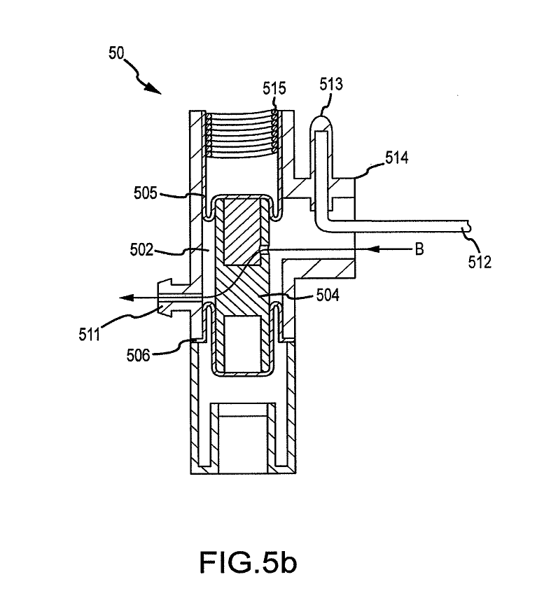

FIGS. 5a and 5b illustrate a device 50 wherein positive pressure ventilation of the patient may be provided in one phase; e.g., when device 50 is being used in association with a CPAP system; and alternative continuous vacuum may be provided in a second phase in which the patient may be isolated from the CPAP system; e.g. when device 50 is being used to apply continuous vacuum to an airway system during CPR in accordance with the invention. Device 50 may comprise a housing 501 that defines a longitudinal lumen 502 and a branch lumen 503 in fluid communication with lumen 502. Piston 504 may be disposed within lumen 502, which may be sealed at the upper section of piston 504 by rolling diaphragm 505 and may be sealed at the lower section of piston 504 by rolling diaphragm 506. The lower section of piston 504 may comprise an internal chamber 507 having an entrance opening 508 at its lower end and an exit opening 509 in its side wall. Lumen 502 may communicate with a CPAP machine (not shown) through connection 510 and may communicate with a vacuum source (not shown) through vacuum connection 511. Oxygen catheter 512, disposed within branch lumen 503, may be connected at one end to an O₂ source (not shown) through connector/valve 513 communicating with branch lumen 503. The opposite end of oxygen catheter 512 may extend the length of a patient's ventilation tube (not shown) so as to direct high velocity O₂ to the patient's respiratory system. The ventilation tube may be connected to lumen 503 through patient connector 514.

Piston 504 and rolling diaphragms 505 and 506 may be moveable between the positions shown in FIGS. 5a and 5b. When positive pressure is present in lumen 502, e.g., when CPAP is being applied to the patient, piston 504 may be moved against biasing spring 515 to the position shown in FIG. 5a. In this position, rolling diaphragm 506 seals lumen 502 above branch lumen 503 and uncovers exit opening 509 so that lumen 502 communicates with branch lumen 503. As illustrated by flow line A in FIG. 5a, this position allows gas under positive pressure to flow into lumen 502 from the ventilator, into chamber 507 through entrance opening 508, into branch lumen 503 through exit opening 509, and then into the patient's ventilation tube connected to branch lumen 503 through connector 514.

When positive pressure is not present in lumen 502, e.g., when the patient is undergoing CPR in accordance with the invention, piston 504 may be moved by the action of biasing spring 515 to the position shown in FIG. 5b. In this position, rolling diaphragm 505 seals lumen 502 at the top of piston 504 and rolling diaphragm 506 seals lumen 502 at the bottom of piston 504. As illustrated by flow line B in FIG. 5b, gas under negative pressure (vacuum) may flow into lumen 502 from the patient's ventilation tube through connector 514, may flow around piston 504, and may exit through vacuum connector 511. Rolling diaphragms 505 and 506 seal the portion of lumen 502 surrounding piston 504 so that ventilator gas leakage into that portion is prevented while vacuum is being applied, thereby isolating the patient from the ventilator gas.

Device 50 may be particularly useful when it is critical to prevent gas applied by the ventilator from reaching the patient during the administration of CPR, e.g., when anesthesia gas is applied to the patient by the ventilator. In addition, device 50 may provide a pressure-balanced system wherein the level of continuous vacuum being applied during CPR according to the invention does not affect the level of pressure required to activate the device when positive pressure ventilation is desired. During CPR in accordance with the invention, a continuous flow of high velocity O₂ may be supplied to the patient's ventilation tube via oxygen catheter 512 and a continuous vacuum may be simultaneously applied as shown in FIG. 5b. Alternatively, e.g. during traditional CPR procedures using positive pressure ventilation, device 50 may allow the supply of O₂ to be turned on or off at connector/valve 513. In other embodiments, device 50 may be used to apply O₂ and a continuous vacuum during CPR for at least 20 seconds according to the invention, but then interspersed with short periods of traditional CPR conducted at atmospheric pressure or with positive pressure ventilation. This combination of CPR methods of the present invention with traditional CPR methods may in some circumstances aid in the ventilation if the patient is found to have inadequate oxygenation.

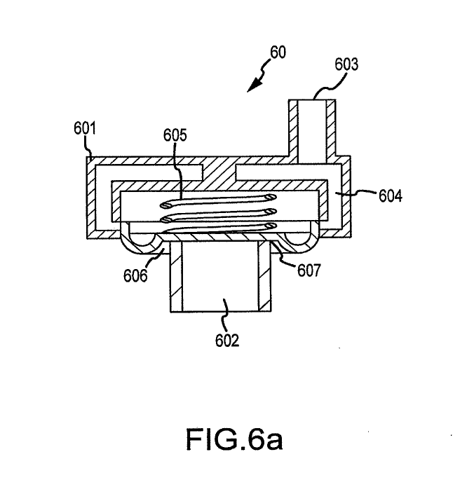

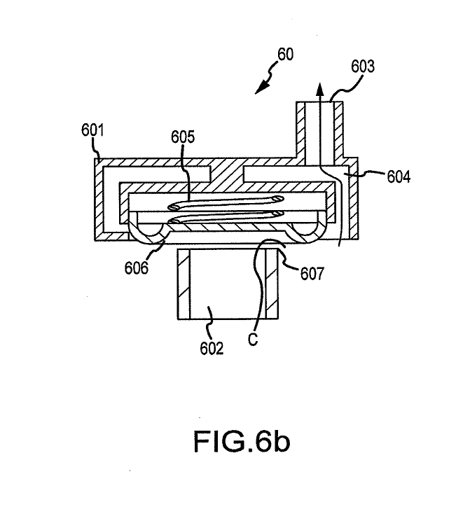

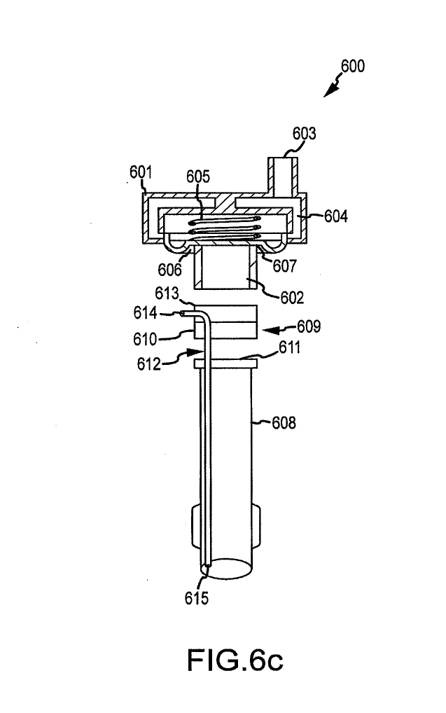

As previously described in connection with FIG. 3, O₂ may be continuously injected into the patient's lungs at a relatively high velocity during continuous CPR chest compressions using apparatus separate from apparatus used to apply and maintain the vacuum to the patient's airway. FIGS. 6a and 6b illustrate a valve 60 that may be used for applying vacuum when the injection of high velocity O₂ is accomplished using a separate device, e.g. an adaptor coupled with ventilator tube such as shown in FIG. 3. Valve 60 may also be useful to help clear CO₂ from the airway each time the chest is compressed while preventing respiratory gases from reentering the patient's respiratory system during the decompression phase of CPR.

FIG. 6c illustrates a resuscitation system 600 comprising a valve 60 coupled with a patient's endotracheal tube 608 through an adaptor 609. Adaptor 609 may comprise a body 610 configured to connect patient lumen 602 with central lumen 611 of endotracheal tube 608. O₂ cannula 612 may enter body 610 through side port 613 and, when adaptor 609 is connected to endotracheal tube 608, may extend the length of central lumen 611. The proximal end 614 of O₂ cannula 612 may be connected to a source of O₂ and the distal end 615 of O₂ cannula 612 may be disposed to inject high velocity O₂ into the patient's lungs.

Valve 60 may comprise a housing 601 including a patient lumen 602 and a vacuum lumen 603 attached to a vacuum source (not shown). The vacuum source may be powered by a small Venturi attached to the O₂ cannula 612 to produce a relatively low level vacuum, or may be an external vacuum source that produces a somewhat higher level of vacuum. Vacuum lumen 603 may be connected to patient lumen 602 through circumferential conduit 604. Biasing spring 605 may be disposed in housing 601 and may be adapted to exert downward force on circumferential sealing gasket 606 so as to keep sealing gasket 606 in the position shown in FIG. 6a. In that position, sealing gasket 606 closes the gap 607 between patient lumen 602 and conduit 604 and prevents respiratory gases from entering or leaving patient lumen 602. For example, each time the chest wall recoils in the decompression phase of CPR chest compressions, sealing gasket 606 occludes patient lumen 602 and thereby allows for the generation and maintenance of decompression phase negative intrathoracic pressure within the patient's airway; provided O₂ flow into the patient's lungs from O₂ cannula 612 is maintained at a rate less than the intrathoracic vacuum generated by the chest recoil.

During the compression phase of CPR chest compressions, the compression forces on the chest may generate a positive intrathoracic pressure which causes respiratory gases such as CO₂ to flow from the patient's lungs and endotracheal tube 608 through patient lumen 602. The intrathoracic positive pressure in combination with the negative pressure generated by the low level vacuum applied through vacuum connection 603 acts to move sealing gasket 606 into the position shown in FIG. 6b, wherein sealing gasket 606 is separated from patient lumen 602. As a result, respiratory gases may be allowed to flow through patient lumen 602, into conduit 604 and out vacuum connection 603, as shown by flow path C in FIG. 6b. Each time sealing gasket 606 is separated from the patient lumen 602 during the compression phase, respiratory gases are sucked out of the trachea, thereby facilitating the efflux of respiratory gases from the patient's lungs. At the end of the compression phase, the absence of positive intrathoracic pressure and the force of biasing spring 605 act to return sealing gasket 606 to the position shown in FIG. 6a, thereby resealing patient lumen 602. In that position, sealing gasket 606 closes the gap 607 between patient lumen 602 and conduit 604 and prevents respiratory gases from entering the patient lumen 602.

In another embodiment, a continuous vacuum may be applied through vacuum connection 603 at a level sufficient to exert an upward force on sealing gasket 606 that is greater than the downward force provided by biasing spring 605. Accordingly, sealing gasket 606 may remain in the open position shown in FIG. 6b as long as such continuous vacuum is being applied at the required level. In this way, a continuous vacuum may be applied to the patient's airway for a sustained period of time through device 60; e.g. for a time period ranging from about 15 seconds to the end of the CPR procedure in accordance with the invention. When the continuous vacuum is removed or reduced; e.g. when it is desired to use device 60 in a conventional CPR procedure, the absence of the greater force provided by the continuous vacuum may allow the force of biasing spring 605 to return sealing gasket 606 to the position shown in FIG. 6a, thereby resealing patient lumen 602.

A variety of airway systems may be modified so as to be suitable for the practice of the invention. In one embodiment, one or more additional lumens may be added within an existing lumen of an airway adjunct specifically to carry O₂ and direct it towards the patient's trachea. The additional lumen(s) may vary in size and design, but the diameter of the lumen(s) will be sufficient to deliver a high O₂ velocity.

Airway adjuncts may be used to protect the lungs from aspiration as well as provide a means to ventilate patients who require assisted ventilation. A number of the previously mentioned airway adjuncts are available for this purpose, including without limitation endotracheal tubes, supraglottic airway devices, Combitubes, obturator airways, laryngeal mask airways, and the like. All of these airway adjuncts may be designed to maintain a seal when positive pressure ventilation is administered to the patient. Some of the airway adjuncts may be further designed to provide a means to prevent gastric contents from entering the lungs; e.g. the airway adjunct may comprise an additional tube portion that can be inserted into the esophagus or stomach. A number of variations are possible; e.g. to enable measuring pressures within the airway adjunct, delivering electrical therapy from the airway adjunct to the body, draining the stomach and gastric content, and the like. Some airway adjuncts are able to be placed in a blinded manner to facilitate ease of insertion. The latter are particularly helpful during the performance of CPR or in treating other life-threatening emergency where endotracheal intubation may be difficult. Most airway adjuncts have one or more cuffed balloons (“cuffs”) to seal off the trachea, the esophagus, the larynx, and other part of the airway tree such that when a positive pressure is delivered to the patient it is directed into the lungs. Dual lumen tubes have also been developed; e.g. to provide ‘jet ventilation’ to suction out mucous and deliver O₂ within the lumen of an endotracheal tube.

Prior to the present invention there has not been a need to seal the patient's airway to allow for the application of a continuous vacuum, nor has there been airway systems adapted to accomplish this result. Standard cuffs are designed to prevent air leaks when positive pressure ventilation is delivered to the patient's airway. Pulling a continuous vacuum by an external means to create a negative intrathoracic pressure in accordance with the invention creates the opportunity for leaks to develop around such standard cuff because the forces on the tube generated by the vacuum may pull the tube inward and create gaps, especially in the nasopharyngeal region of the airway. In one embodiment, the present invention provides a means to easily and effectively generate and maintain a continuous vacuum without air leaks to the outside. The present invention is thereby useful for optimizing new therapies for the treatment of various conditions that take advantage of the beneficial effects of negative intrathoracic pressure. Such conditions include without limitation cardiac arrest, shock, stroke, brain injury and other states of low blood circulation.

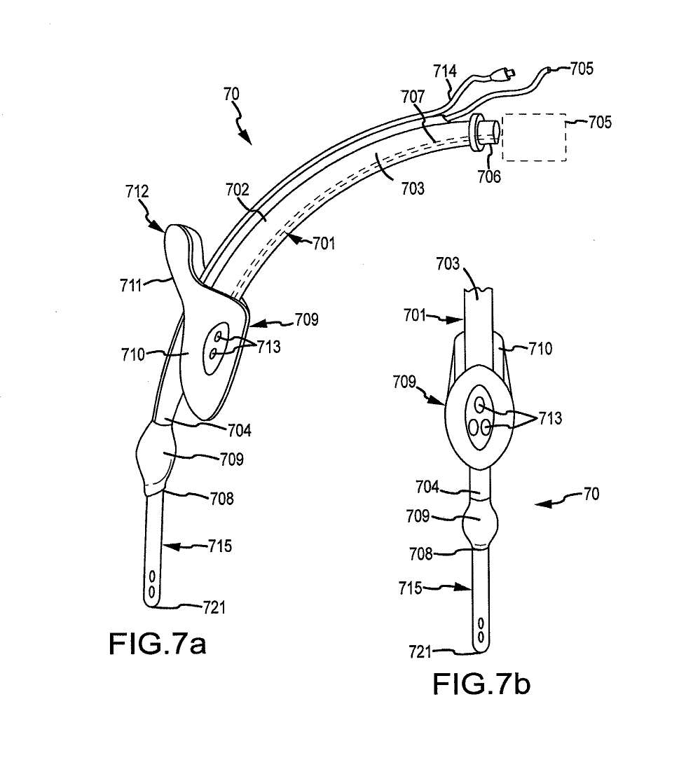

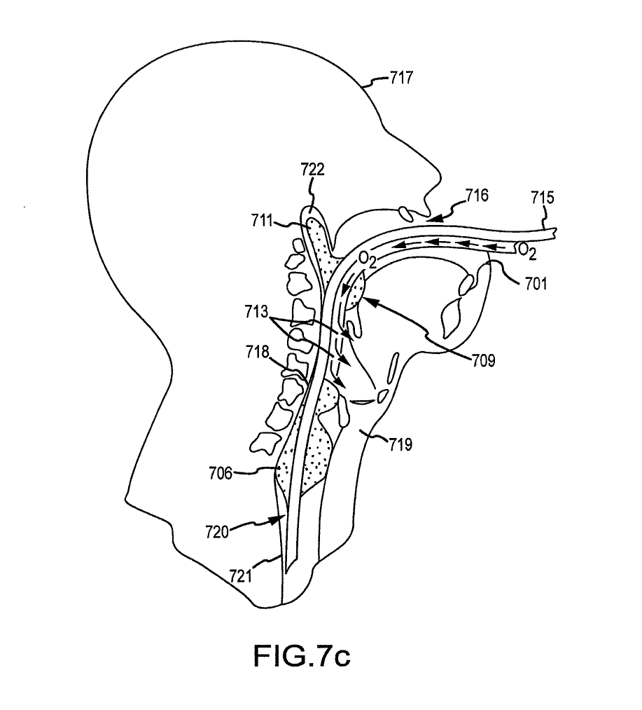

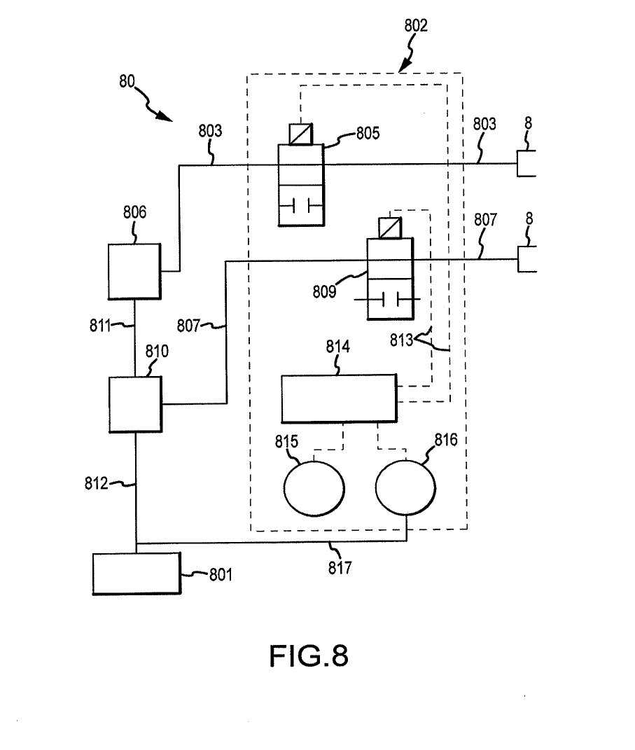

The following description refers to FIGS. 7a-7c, and sets forth one non-limiting example of a novel airway system that may be used in accordance with the present invention, as well as prior art therapies wherein the development and maintenance of a negative intrathoracic pressure may be beneficial; e.g. methods, systems and devices that utilize ITDs as described in the aforementioned and previously incorporated U.S. patents.

FIG. 7a shows a side view of a novel locking supraglottic airway (LSA) adjunct 70 that may be used in the practice of the present invention, and FIG. 7b shows a view of LSA 70 on a plane perpendicular to the view of FIG. 7a. FIG. 7c shows a sagittal view of LSA 70 interfaced with a patient's airway 716. SLA 70 may comprise an airway tube 701 having a central lumen 702 with a proximal supraglottal section 703 and a distal esophageal section 704. System 705 may be attached to proximal end 706 of airway tube 701. System 705 may be any of the previously described systems of the present invention that comprise means for applying a continuous vacuum and means for delivering high velocity O₂, either integrated into a single device or separated into more than one device. For example, system 705 may comprise one or more of the devices shown in FIGS. 2-6. Accordingly, a continuous vacuum may be applied by system 705 to central lumen 702 of airway tube 701 and O₂ may be delivered by system 704 through one or more O₂ cannula 707 disposed in supraglottal section 703 of central lumen 702. Distal end 708 of esophageal section 704 may be surrounded by esophageal cuff 706.

Laryngeal cuff 709 may be positioned so as to surround airway tube 701 at the distal end of supraglottal section 703. Laryngeal cuff 709 may comprise cuff body 710 and nasopharyngeal extension 711, which may include a thickened wall portion 712 that serves as a stiffener. Laryngeal cuff 709 and esophageal cuff 706 may comprise balloons that could be inflated with a syringe. In one embodiment, both cuffs may be inflated with a single syringe, or a separate syringe may be used to inflate each balloon. O₂ cannula 707 may extend the length of supraglottal section 703 from proximal end 706 to laryngeal cuff 709, where O₂ cannula 707 may terminate as one or more ventilation ports 713 in laryngeal cuff 709. LSA 70 may also comprise pilot tube 714 attached to and extending the length of airway tube 701. Pilot tube 714 is adapted to receive orogastric tube 715.

LSA 70 may be interfaced with airway 716 of patient 717 as shown in FIG. 7c. For a more complete understanding, various parts of the anatomy of patient 717 are labeled in FIG. 7c without reference numerals. Whereas even skilled individuals may have difficulty easily and reliably placing prior art supraglottic airways, orogastric tubes are generally easy to pass into a patient's airway. Accordingly, orogastric tube 715 may be first passed into esophagus 718 of patient 717. Once orogastric tube 715 is inserted and advanced into esophagus 718, pilot tube 714 may be slid over orogastric tube 715 so that orogastric tube 715 may be used as a guide to facilitate the proper placement of LSA 70 in esophagus 718 as well as the proper seating of laryngeal cuff 709 and esophageal cuff 706. For example, LSA 70 may be advanced down along the length of orogastric tube 715 until distal end 720 of pilot tube 714 stops short of the distal tip 721 of orogastric tube 715, as shown in FIG. 7c. Also as shown in FIG. 7c, esophageal cuff 706 and body 710 of laryngeal cuff 709 may be positioned to seal off esophagus 718, and nasopharyngeal extension 711 of laryngeal cuff 709 may be positioned to seal off the nasopharyngeal section 722 of airway 716. As one example of a benefit derived from this facilitated placement, LSA 70 may be blindly placed by a rescuer performing CPR under field conditions without stopping CPR chest compressions. The improved seating of laryngeal cuff 709, esophageal cuff 706 and nasopharyngeal extension 711 may also assure a more complete sealing of airway 716 when a vacuum is generated and maintained in trachea 719 of patient 717, below supraglottic section 703 of LSA 70.

In one embodiment of the invention, high velocity O₂ may be injected from O₂ cannula 707 directly into the trachea of patient 717 through ventilation ports 713 by positioning laryngeal cuff 709 so that high velocity O₂ exiting from ventilation ports 713 are physically directed toward the central lumen of the patient's trachea and main stem bronchi, as shown in FIG. 7c. In another embodiment, a continuous vacuum may be applied to airway tube 701 at a level sufficient to generate a negative pressure in the trachea of patient 717, which as previously described, is facilitated by the improved seating of laryngeal cuff 709, esophageal cuff 706 and nasopharyngeal extension 711 provided by LSA 70.

LSA 70 provides novel means for interfacing with the patient's airway in the practice of the invention. LSA 70 is designed with special nasopharyngeal appendage 711, which seals off the nasopharyngeal passageway 722 when inserted into the patient's airway 716. As shown in FIG. 7a, LSA 70 may in one optimal embodiment have a bend to direct the distal tip 721 into the esophagus when it is inserted blindly. Distal tip 721 may be used to stabilize LSA 70 and orogastric tube 715 may optionally serve as a conduit to drain gastric contents.

As previously described, laryngeal cuff 709 may be a small balloon that has a unique feature, nasopharyngeal appendage 711, that serves to seal the nasopharyngeal region of the airway concurrently with the laryngeal-pharyngeal cavity. As a result, the patient's airway may be sealed and the airway tube may be stabilized so that it is more difficult for the airway tube to advance into the airway and leak when a continuous vacuum is drawn in the thorax relative to the atmosphere. In addition, LSA 70 may prevent gastric contents from being sucked into the patient's lungs. In contrast with prior art airway adjuncts that have inflatable cuffs to help seal off the airway and allow for the delivery of a positive pressure breath, LSA 70 is designed to assure the maintenance of continuous vacuum in the patient's airway and to continuously deliver high velocity O₂ to the patient in accordance with the present invention. In addition, SLA 70 may provide a means for rapidly placing an airway adjunct blindly by the rescuer performing CPR, without stopping chest compressions, and may also protect against pulmonary aspiration. Laryngeal cuff 709 and nasopharyngeal extension 711, along with esophageal cuff 709, may assure a tight seal, even when a vacuum is generated below the position of SLA 70 in the patient's airway. SLA 70 also may provide an optional means to cannulate and/or suction the stomach through orogastric tube 715.

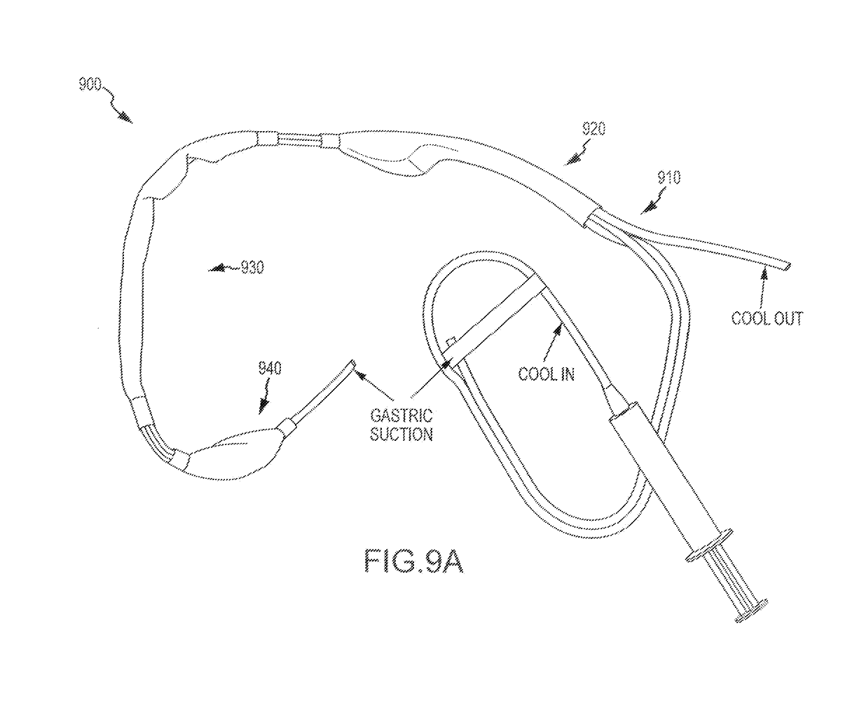

FIG. 8 is a block diagram of an apparatus 80 that may be used in one embodiment of the invention suitable for providing intermittent or continuous O₂ delivery and continuous or intermittent vacuum to a patient 801. Apparatus 80 may also be used to provide intermittent positive airway pressure to patient 801. It should be understood that all of the components of apparatus 80 shown in FIG. 8 may not be required for apparatus 80 to function, and merely represent one example of a suitable apparatus.

Referring to FIG. 8, controller 802 of apparatus 80 may be connected to gas line 803, which runs from O₂ source 804 through control valve 805 to continuous positive airway pressure or bi-level positive pressure (CPAP/BiPAP) device 806. Controller 802 may also be connected to vacuum line 807, which runs from vacuum source 808 through control valve 809 to impedance threshold device or intrathoracic pressure regulator (ITD/ITPR) 810. CPAP/BiPAP device 806 and ITD/ITPR device 810 may be connected by line 811 and ITD/ITPR device 810 may be connected to patient 801 by line 812. Control valve 805 and 809 may be electrically controlled by microcontroller 811 through wires 813 so as to regulate the application of O₂ and vacuum and the positive airway pressure to patient 801. Microcontroller 811 may be adjusted using timer 813, CO₂ detector 814 that measures the amount of CO₂ present in the respiratory system of patient 801, or a using a variety of other instruments for determining the proper application of O₂ and vacuum, or positive airway pressure, to patient 817, for example, an airway pressure sensor.

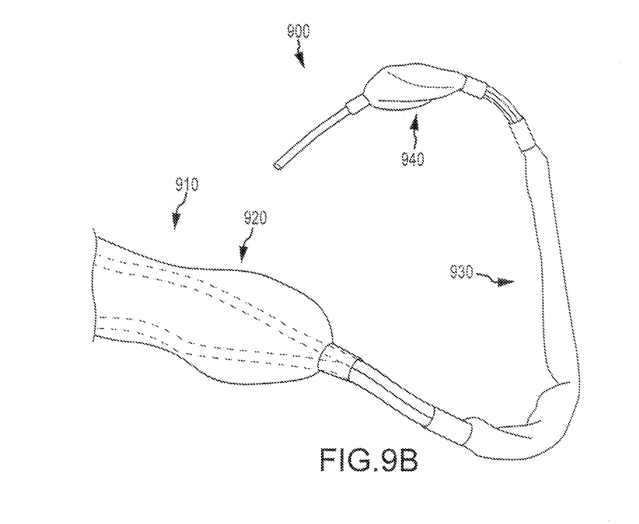

FIGS. 9A and 9B show aspects of an airway adjunct system 900, according to embodiments of the present invention. Airway adjunct system 900 can be used during administration of a resuscitative procedure to a patient. As shown here, airway adjunct system 900 includes a support assembly 910 defining an intrathoracic pressure monitoring lumen, a fluid delivery lumen through which a cooling fluid may be delivered, a fluid return lumen through which a cooling fluid may be returned, and an orogastric lumen. Treatment of the patient with cooling fluid can provide a preservative effect on the patient's heart tissue, as well as the patient's cerebral and brain tissue. The support assembly 910, and other components of the system, can be configured to present an oval profile, which can prevent or inhibit the system from unwanted rotation within the patient. The support assembly can also define a lumen for passage of electrical stimulation and recording wires. Such wires can be coupled with sensing elements for monitoring the patient's heartbeat, or with pacing or stimulation elements for restoring cardiac rhythm in the patient. Airway adjunct system 900 also includes an intrathoracic pressure monitoring mechanism (not shown) in fluid communication with the intrathoracic pressure monitoring lumen. The intrathoracic pressure monitoring mechanism monitors pressure within the intrathoracic pressure regulation lumen during administration of the resuscitative procedure to the patient. Additionally, airway adjunct system 900 includes a laryngeal cuff assembly 920 coupled with the support assembly 910. The laryngeal cuff assembly 920 can be positioned within the patient's supraglottic airway, for example by maneuvering the support assembly 910. In some cases, the laryngeal cuff assembly 920 at least partially isolates and seals a portion of the supraglottic airway of the patient. The laryngeal cuff assembly 920 can operate to assist in positioning a distal section of the intrathoracic pressure monitoring lumen in fluid communication with the patient's trachea, during administration of the resuscitative procedure to the patient. Airway adjunct system 900 also includes an esophageal cuff assembly 930 coupled with the support assembly 910 distal to the laryngeal cuff assembly 920. The esophageal cuff assembly 930 can interface with the esophagus of the patient during administration of the resuscitative procedure to the patient. In use, the esophageal cuff assembly 930 can provide a platform against which the heart may be compressed against, when positioned accordingly within the patient's anatomy. The esophageal cuff assembly 930 may also include an electrode assembly for monitoring or stimulating electrical activity within the patient, such as cardiac electrical activity. Further, airway adjunct system 900 includes a gastric cuff assembly 940 coupled with the support assembly 910 distal to the esophageal cuff assembly 930. The gastric cuff assembly operates to assist in positioning a distal section of the orogastric lumen in fluid communication with the patient's stomach, so that the orogastric lumen can facilitate removal of gastric contents from the patient during administration of the resuscitative procedure to the patient. For example, a gastric balloon can operate to perform an anchoring function. Airway adjunct system 900 also includes a fluid passage circuit defined at least in part by the esophageal cuff assembly 930 and the gastric cuff assembly 940 and in fluid communication with the fluid delivery lumen and the fluid return lumen of the support assembly 910. The fluid passage circuit can be used to circulate cooling fluid through the esophageal cuff assembly 930 and the gastric cuff assembly 940, for example. Hence, the esophageal cuff assembly 930 and the gastric cuff assembly 940 can be inflated in unison. In some cases, the fluid passage circuit can include one or more valves, such as one-way or two-way valves. A syringe can be used to introduce cooling fluid into the fluid delivery lumen.

According to embodiments of the present invention, intrathoracic pressure regulation can occur during active chest compression and decompression, and can be regulated by an intrathoracic pressure regulation mechanism such as an Impedance Threshold Device (ITD) or an Intrathoracic Pressure Regulator (ITPR). Both ITD and ITPR mechanisms can be used to decrease intrathoracic pressure or otherwise facilitate negative airway pressure in a patient, so as to enhance circulation. Operation of an ITD mechanism involves vacuum associated with recoil, and operation of an ITPR mechanism involves the active application of a vacuum. Both ITD and ITPR mechanisms can be used to lower intrathoracic pressure during the chest wall recoil phase of CPR, thereby enhancing the transfer of blood from outside the thorax into the right heart. According to some embodiments, an ITD valve system can be configured to prevent respiratory gas flow to the person's lungs during the decompression phase until a negative airway pressure achieved equals the opening pressure of the valve system, and an ITPR valve system can be used to withdraw air from the lungs via an active vacuum source until a negative airway pressure is achieved. Often, ITD and ITPR procedures can be used independently, such that a patient may be treated with either an ITD mechanism or an ITPR mechanism.

Airway adjunct systems can include intrathoracic pressure monitoring mechanisms and intrathoracic pressure regulation mechanisms. In some cases, monitoring and regulation mechanisms are integral with each other. In some cases, monitoring and regulation mechanisms are separate parts of the system. Optionally, monitoring and regulation mechanisms can either be operated in a coordinated fashion, or independently from one another. In some cases, monitoring mechanisms can be used to perform pressure regulation operations, and regulation mechanisms can be used to perform pressure monitoring operations.

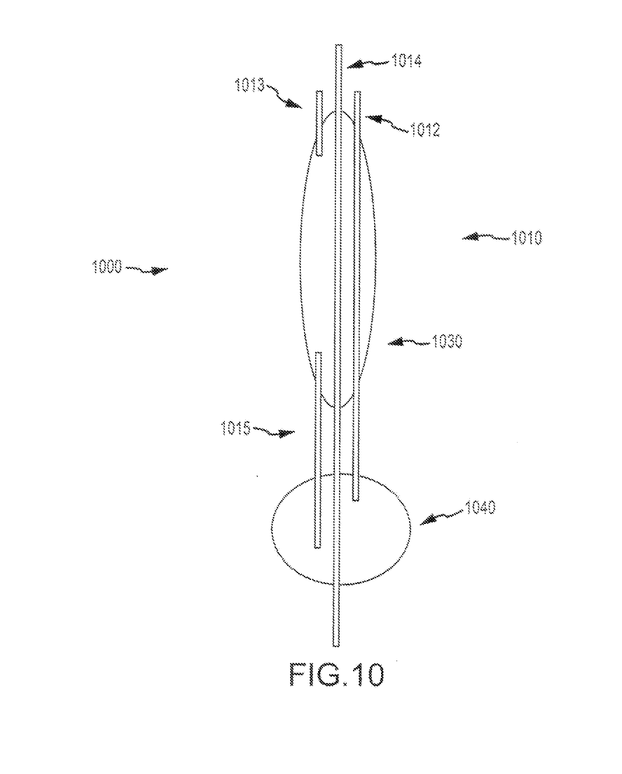

FIG. 10 schematically illustrates aspects of an airway adjunct system 1000, according to embodiments of the present invention. Airway adjunct system 1000 can be used during administration of a resuscitative procedure to a patient. As shown here, airway adjunct system 1000 includes a support assembly 1010 defining an intrathoracic pressure monitoring lumen, a fluid delivery lumen 1012 through which a cooling fluid may be delivered, a fluid return lumen 1013 through which a cooling fluid may be returned, and an orogastric lumen 1014. Airway adjunct system 1000 also includes an intrathoracic pressure monitoring mechanism (not shown) in fluid communication with the intrathoracic pressure monitoring lumen. The intrathoracic pressure monitoring mechanism monitors pressure within the intrathoracic pressure regulation lumen during administration of the resuscitative procedure to the patient. Additionally, airway adjunct system 1000 includes a laryngeal cuff assembly (not shown) coupled with the support assembly 1010. The laryngeal cuff assembly can be positioned within the patient's supraglottic airway, for example by maneuvering the support assembly 1010. In some cases, the laryngeal cuff assembly at least partially isolates and seals a portion of the supraglottic airway of the patient. The laryngeal cuff assembly can operate to assist in positioning a distal section of the intrathoracic pressure monitoring lumen in fluid communication with the patient's trachea, during administration of the resuscitative procedure to the patient. Airway adjunct system 1000 also includes an esophageal cuff assembly 1030 coupled with the support assembly 1010 distal to the laryngeal cuff assembly. The esophageal cuff assembly 1030 can interface with the esophagus of the patient during administration of the resuscitative procedure to the patient. Further, airway adjunct system 1000 includes a gastric cuff assembly 1040 coupled with the support assembly 1010 distal to the esophageal cuff assembly 1030. The gastric cuff assembly operates to assist in positioning a distal section of the orogastric lumen 1014 in fluid communication with the patient's stomach, so that the orogastric lumen 1014 can facilitate gastric decompression or removal of gastric contents from the patient during administration of the resuscitative procedure to the patient. Airway adjunct system 1000 also includes a fluid passage circuit defined at least in part by the esophageal cuff assembly 1030 and the gastric cuff assembly 1040 and in fluid communication with the fluid delivery lumen and the fluid return lumen of the support assembly 1010. The fluid passage circuit can be used to circulate cooling fluid through the esophageal cuff assembly 1030 and the gastric cuff assembly 1040, for example. Optionally, the fluid passage circuit can include a transfer passage 1015 through which fluid such as cooling fluid may pass between the esophageal cuff assembly 1030 and the gastric cuff assembly 1040.

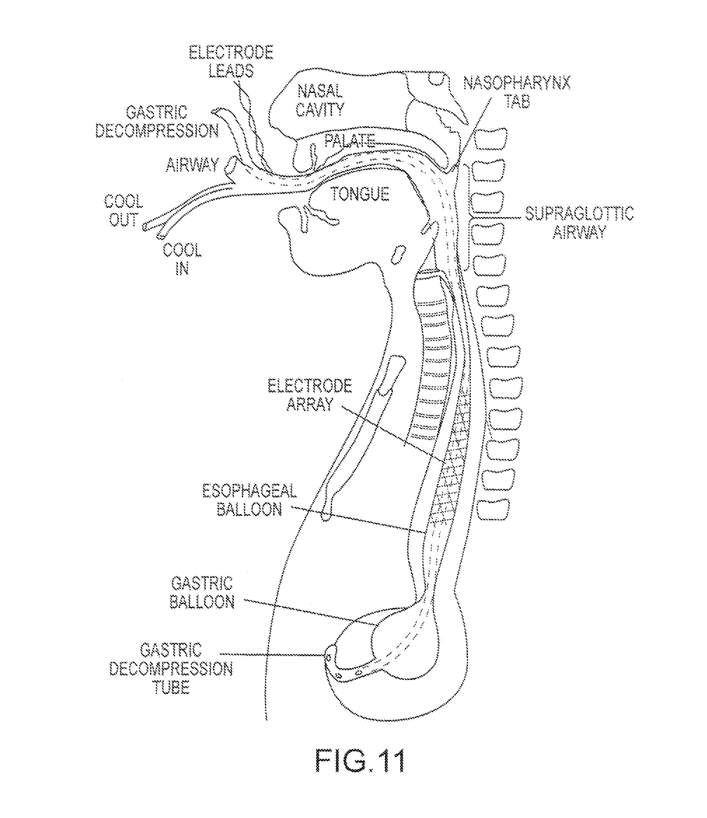

FIG. 11 shows placement of an airway adjunct system within a patient's anatomy, according to embodiments of the present invention.

According to some embodiments, an esophageal cuff assembly and a gastric cuff assembly can be contiguous. Typically, the esophageal cuff assembly engages the esophagus of the patient and the gastric cuff assembly engages the stomach of the patient so as to assist in securing the laryngeal cuff assembly in place within the patient during administration of the resuscitative procedure to the patient. The laryngeal cuff assembly can include a nasopharyngeal extension to help further secure the system at a desired location or position within the patient. In some cases, the laryngeal cuff assembly includes an elastomeric gel body that is shaped to interface with contours of the patient's airway. In some cases, the laryngeal cuff assembly defines an inflatable lumen, and the support assembly defines an inflation lumen that is in fluid communication with the inflatable lumen of the laryngeal cuff. Optionally, an airway adjunct resuscitation system can include a bite block mechanism coupled with the support assembly. In some cases, an esophageal cuff assembly has a length of at least 3 cm. In some cases, an esophageal cuff assembly has a length within a range from about 20 cm to about 30 cm. According to some embodiments, the esophageal cuff assembly includes an inlet port in fluid communication with the fluid delivery lumen and an outlet port in fluid communication with the fluid return lumen. Cooling fluid can be circulated from the inlet tubing into the gastric balloon, and through a connecting tubing which crosses the gastroesophageal junction and serves as an inlet to the esophageal balloon. The outlet port of the esophageal balloon can be at the upper end of the balloon and connect to the outlet tubing exiting the body via the support assembly. According to some embodiments, a laryngeal cuff assembly can define a laryngeal cuff lumen and the esophageal cuff assembly can define an esophageal cuff lumen, and the laryngeal cuff lumen can be in fluid communication with the esophageal cuff lumen.

In some cases, the laryngeal cuff assembly includes a laryngeal cuff balloon and the esophageal cuff assembly includes an esophageal cuff balloon. The laryngeal cuff balloon can be contiguous with the esophageal cuff balloon, such that pressure, volume, or both can be redistributed between the laryngeal cuff balloon and the esophageal cuff balloon when pressure is applied to or released from the patient's sternum during administration of the resuscitative procedure to the patient when the resuscitative procedure comprises administration of external chest compressions. In some cases, the esophageal balloon is not contiguous with the supraglottic balloon if there is a cooling system and if the supraglottic cuff is made of elastomeric gel.

According to some embodiments, an esophageal cuff assembly can provide a platform against which the patient's heart may be compressed during administration of the resuscitative procedure to the patient when the resuscitative procedure comprises administration of external chest compressions. Optionally, the esophageal cuff assembly can include a stimulation assembly. In some cases, the stimulation assembly includes a cardiac stimulation mechanism. In some cases, the cardiac stimulation mechanism includes a cardiac pacing electrode. In some cases, the cardiac stimulation mechanism includes a cardiac defibrillation electrode. According to some embodiments, the esophageal cuff assembly includes a monitoring assembly. Optionally, the monitoring assembly can include a cardiac monitoring mechanism. In some cases, a support assembly further defines an esophageal cuff assembly auxiliary component lumen. In some cases, an airway adjunct resuscitation system can include a connectivity mechanism disposed within the auxiliary component lumen, the esophageal cuff assembly can include a stimulation assembly, and the connectivity mechanism can be coupled with the stimulation assembly. An airway adjunct resuscitation system can also have a connectivity mechanism disposed within the auxiliary component lumen. The esophageal cuff assembly can include a monitoring assembly, and the connectivity mechanism can be coupled with the monitoring assembly. According to some embodiments, an esophageal cuff assembly defines an esophageal cuff lumen and the gastric cuff assembly defines a gastric cuff lumen, and the esophageal cuff lumen is in fluid communication with the gastric cuff lumen. The esophageal cuff assembly can present an oval shape. Some airway adjunct resuscitation systems may include an esophageal manometer that monitors fluid pressure at a location within the airway adjunct resuscitation system. Some airway adjunct resuscitation systems may include an esophageal manometer that monitors fluid pressure at a location within the patient. An esophageal manometer can be integral with an esophageal balloon, and may operate to monitor the pressure of cooling within the treatment system, for example during the application of a chest compression protocol. Optionally, an esophageal cuff assembly may include a heat conducting material. In some instances, an esophageal cuff assembly can include a non-compressible and non-expansible material, such as Mylar, that resists rupture during administration of the resuscitative procedure to the patient when the resuscitative procedure involves administration of external chest compressions. In some instances, an airway adjunct resuscitation system can include one or more physiological sensors. In some instances, an airway adjunct resuscitation system can include a communication mechanism, such as a Bluetooth device, an RF communication device, an antenna, or the like. In some instances, an airway adjunct resuscitation system can include a controlled valve regulation system in operative association with the intrathoracic pressure regulation mechanism.

Embodiments of the present invention further encompass methods of administering a resuscitative procedure to a patient. Exemplary methods may include engaging an airway adjunct resuscitation system with the patient. The airway adjunct resuscitation system can include a support assembly defining an intrathoracic pressure monitoring lumen, an intrathoracic pressure monitoring mechanism in fluid communication with the intrathoracic pressure monitoring lumen, a laryngeal cuff assembly coupled with the support assembly, an esophageal cuff assembly coupled with the support assembly distal to the laryngeal cuff assembly, and a gastric cuff assembly coupled with the support assembly distal to the esophageal cuff assembly. Methods may further include positioning the laryngeal cuff assembly within the patient's supraglottic airway, positioning a distal section of the intrathoracic pressure regulation lumen in fluid communication with the patient's trachea, isolating and sealing a portion of the supraglottic airway of the patient with the laryngeal cuff assembly, placing the esophageal cuff assembly at the esophagus of the patient, placing the gastric cuff assembly at the stomach of the patient, and monitoring pressure within the intrathoracic pressure monitoring lumen with the intrathoracic pressure monitoring mechanism. Methods may also include lifting the patient's heart anteriorly with the esophageal cuff assembly in conjunction with application of a CPR chest compression, so as to increase compression of the patient's heart between the patient's sternum and esophagus. Some methods include flowing an intrathoracic cooling fluid through the esophageal cuff assembly, the gastric cuff assembly, or both. Methods may also include modulating or regulating intrathoracic pressure in the patient with an intrathoracic pressure regulation mechanism, for example to increase circulation within the patient. During the administration of treatment methods, the patient may be disposed in a prone position, a supine position, a standing position, a sitting position, or a sideways lying position. Treatments may also include engaging a bite block mechanism of the airway adjunct resuscitation system with the patient's mouth. Some treatment methods include removing gastric contents from the patient via an orogastric lumen defined by the support assembly of the airway adjunct resuscitation system. Methods may also include monitoring a physiological parameter with a physiological sensor of the airway adjunct resuscitation system. In some cases, methods may include monitoring a transthoracic impedance parameter of the patient with a physiological sensor of the airway adjunct resuscitation system. Optionally, methods may include assessing cardiac output in the patient based on the transthoracic impedance parameter. In some instances, methods may include administering a pacing treatment to the patient's heart based on the transthoracic impedance parameter. Further, methods may include administering a defibrillation treatment to the patient's heart based on the transthoracic impedance parameter, or administering repeated chest compressions to the patient. An intrathoracic pressure regulation mechanism can have a sequential valve system configured to administer no inspiratory resistance for a positive pressure ventilation, no expiratory resistance, an optional PEEP, or a preset or variable resistance to inflow of respiratory gases. In some cases, an intrathoracic pressure regulation mechanism can be configured to generate a continuous negative intrathoracic pressure with intermittent positive pressure ventilation. Optionally, methods may include monitoring an end tidal CO₂ level in the patient with an end tidal CO₂ sensor of the airway adjunct resuscitation system, and adjusting the resuscitative procedure based on the end tidal CO₂ level.

Embodiments of the present invention also encompass methods for treating or evaluating a patient suffering from or at risk of developing a condition selected from the group consisting of heart failure, cardiac arrest, sepsis, shock, acute respiratory distress syndrome, polytrauma, head disease, elevated hepatic or portal vein pressures, bleeding during abdominal, head and neck surgery, and insufficient circulation during open heart surgery. Exemplary methods may include engaging an airway adjunct resuscitation system with the patient, where the airway adjunct resuscitation system includes a support assembly defining an intrathoracic pressure monitoring lumen, an intrathoracic pressure monitoring mechanism in fluid communication with the intrathoracic pressure monitoring lumen, a laryngeal cuff assembly coupled with the support assembly, an esophageal cuff assembly coupled with the support assembly distal to the laryngeal cuff assembly, and a gastric cuff assembly coupled with the support assembly distal to the esophageal cuff assembly. Further, methods may include positioning the laryngeal cuff assembly within the patient's supraglottic airway, positioning a distal section of the intrathoracic pressure regulation lumen in fluid communication with the patient's trachea, isolating and sealing a portion of the supraglottic airway of the patient with the laryngeal cuff assembly, placing the esophageal cuff assembly at the esophagus of the patient, placing the gastric cuff assembly at the stomach of the patient, and monitoring pressure within the intrathoracic pressure monitoring lumen with the intrathoracic pressure monitoring mechanism, so that the patient's trachea is exposed to the monitored pressure within the intrathoracic pressure monitoring lumen.

Embodiments of the present invention further encompass methods for treating or evaluating a patient suffering from or at risk of developing low circulation. Exemplary methods may include engaging an airway adjunct resuscitation system with the patient. The airway adjunct resuscitation system can include, for example, a support assembly defining an intrathoracic pressure monitoring lumen, an intrathoracic pressure monitoring mechanism in fluid communication with the intrathoracic pressure regulation lumen, a laryngeal cuff assembly coupled with the support assembly, an esophageal cuff assembly coupled with the support assembly distal to the laryngeal cuff assembly, and a gastric cuff assembly coupled with the support assembly distal to the esophageal cuff assembly. Methods may also include positioning the laryngeal cuff assembly within the patient's supraglottic airway, positioning a distal section of the intrathoracic pressure monitoring lumen in fluid communication with the patient's trachea, isolating and sealing a portion of the supraglottic airway of the patient with the laryngeal cuff assembly, placing the esophageal cuff assembly at the esophagus of the patient, placing the gastric cuff assembly at the stomach of the patient, and expanding the esophageal cuff assembly so as to anteriorly move or provide support for the heart within the patient's body. Further, methods may include administering of external chest compressions to the patient so as to compress the patient's heart against the esophageal cuff assembly, and monitoring pressure within the intrathoracic pressure monitoring lumen with the intrathoracic pressure monitoring mechanism, so that the patient's trachea is exposed to the monitored pressure within the intrathoracic pressure monitoring lumen.

Embodiments of the present invention additionally encompass methods for providing a cooling treatment to a patient. Exemplary methods may include engaging an airway adjunct resuscitation system with the patient. An airway adjunct resuscitation system may include, for example, a support assembly defining an intrathoracic pressure monitoring lumen, an intrathoracic pressure monitoring mechanism in fluid communication with the intrathoracic pressure monitoring lumen, a laryngeal cuff assembly coupled with the support assembly, an esophageal cuff assembly coupled with the support assembly distal to the laryngeal cuff assembly, and a gastric cuff assembly coupled with the support assembly distal to the esophageal cuff assembly. Further, methods may include positioning the laryngeal cuff assembly within the patient's supraglottic airway, positioning a distal section of the intrathoracic pressure monitoring lumen in fluid communication with the patient's trachea, isolating and sealing a portion of the supraglottic airway of the patient with the laryngeal cuff assembly, placing the esophageal cuff assembly at the esophagus of the patient, placing the gastric cuff assembly at the stomach of the patient, and introducing a cooling fluid into the esophageal cuff assembly, the gastric cuff assembly, or both.

Embodiments of the present invention encompass systems and methods that provide an esophageal balloon for cooling or warming a patient, in conjunction with means to ventilate a patient and also, in some modes of operation, means to apply the use of impedance threshold device technology. Exemplary embodiments provide a device that include a number of components attached together in a single tube designed to provide a means to rapidly, reliably, and safely secure a supraglottic airway for positive pressure ventilation, to provide a means to seal the airway to allow for developing and maintaining negative intrathoracic pressure and to reduce the risk of respiration, a means to stabilize the device with an esophageal balloon, a means to augment circulation with an impedance threshold device incorporated within the tube, a and means for management of stomach contents through the tube. Additional optional elements include: a means to cool the body by circulation of cold fluid through the esophageal and optional gastric balloon, a means to detect a number of physiological parameters, a means to deliver therapy to the patient, including but not limited to drug therapy and electrical stimulation, and a means to provide feedback and instructions to rescue personnel to maintain quality of care. Exemplary devices and techniques can utilize the patient's anatomy from the oropharynx to the stomach to deliver life-saving therapies critical for resuscitation during states of severe hypotension and cardiac arrest.

Securing the airway during CPR and other resuscitative procedures is often an important element in the care of patients in extremis, including cardiac arrest. Embodiments of the present invention provide for ease of insertion, improved control of gastric contents, and reduced risk of displacement, as compared with many currently available products. Further, embodiments of the present invention are well suited for delivering a number of different therapies simultaneously, which can be particularly beneficial for treating patients in cardiac arrest. For example, embodiments of the present invention allow the operator or physician to efficiently secure the patient's airway, to manage the gastric contents, to isolate the airway for aspiration protection, to control intrathoracic pressure during resuscitation, to provide a platform or mechanical advantage during chest compression, to provide continuous core cooling, and to provide for the monitoring of intrathoracic pressure and other physiological parameters. System embodiments can be inserted into the patient easily and reliably by an operator.

In some instances, systems provide a single device having a supraglottic airway isolation and sealing device that is flexible, durable, and conforms to the anatomy of the supraglottic space. Systems may also include a nasopharyngeal anchor or laryngeal cuff, and can have the capacity for replacement with an endotracheal (ET) tube using a guide and guiding port. Some systems may include an integral bite block, and can provide variable seal pressure during the administration of chest compressions. Often, one or more elements of a system present an anatomically compatible shape or contour. Systems may include an esophageal balloon with access port(s) for infusion of liquids and gases. In some instances, esophageal balloons can have a minimal length of about 3 cm, and optimal length of between about 20 cm and about 30 cm. Systems may further include a connection between an supraglottic airway cuff and an esophageal balloon. In some cases, systems include an esophageal balloon having a contiguous gastric balloon. An esophageal balloon can have an oval shape.

According to some embodiments, systems include an integral impedance threshold device that provides a sequential valve system for no inspiratory resistance for positive pressure ventilation, no expiratory resistance or optional PEEP, and resistance to inflow of respiratory gases with a threshold device either preset or variable. Integral impedance threshold devices can also provide an optional means to generate a continuous negative intrathoracic pressure with intermittent positive pressure ventilation. Exemplary systems may also include an orogastric tube.

In some embodiments, systems include contiguous balloons, which may involve a connection between an esophageal balloon and a cuff of a supraglottic airway, that allow for pressure and/or volume redistribution when pressure is applied to the sternum during external chest compressions of a CPR treatment. Systems may also include an end tidal CO₂ sensor, flashing lights and other user feedback tools, an esophageal manometer, an esophageal balloon fluid circulator system and elements in the balloon design for fluid exchange to optimize heat transfer, electrodes for pacing, defibrillation, and monitoring, and other physiological sensors. Systems may also include Blue tooth communication mechanisms or other RF means to communicate remotely to an external receiver, and an antenna to receive input. In some cases, systems include a solenoid based, software controlled valve regulation system for an impedance threshold valve.

Treatment methods can be administered to enhance circulation by altering the way the heart is compressed during CPR. For example, treatments can involve lifting the heart anteriorly with an esophageal balloon, or providing a platform for cardiac compression using the esophageal balloon, resulting in more compression of the heart between the sternum and the esophagus. Some treatment methods can be administered for intrathoracic cooling, or for enhancing circulation via impedance technology. Exemplary methods also provide for rapid and reliable airway establishment independent of patient position or if the patient is receiving chest compression for CPR. Methods may also involve securing a tube of the system inside the patient's mouth, and removing gastric contents from the patient. In some cases, methods include sensing physiological parameters, such as the use of transthoracic impedance to assess cardiac output, to pace the heart, and to defibrillate.

According to some embodiments, treatment systems may include a nasopharyngeal extension which can operate to help secure the system in place within the patient's anatomy. Optionally, or in place thereof, gastric and esophageal balloons can help to secure the system in place within the patient's anatomy. Systems may include an inflatable supraglottic cuff that is separate from the gastric and esophageal balloons, and the gastric and esophageal balloons can be part of a cooling system. In some cases, a supraglottic cuff can include a gel material. An orogastric tube can operate as a guide for the system, and can also be used to decompression the patient's stomach. Cooling lines may run with or in the orogastric tube, in some cases.

Exemplary embodiments encompass airway adjunct systems which provide a supraglottic airway mechanism, optionally in operative association with an ITD mechanism. An on/off switch may be included with the ITD mechanism. An esophageal balloon can operate to stabilize the system, to increase CPR efficiency but propping up the patient's heart, and to cool the patient. The airway adjunct system can also include mechanisms for monitoring airway pressure, temperature, electrical signals, and other physiological parameters.

Embodiments of the present invention have now been described in detail for the purposes of clarity and understanding. However, it will be appreciated that certain changes and modifications may practiced within the scope of the appended claims.

1. An airway adjunct resuscitation system for use during administration of a resuscitative procedure to a patient, the system comprising:
a support assembly defining an intrathoracic pressure monitoring lumen, a cooling fluid delivery lumen, a cooling fluid return lumen, and an orogastric lumen, wherein the orogastric lumen is adapted for use in removing gastric contents from the patient during administration of the resuscitative procedure to the patient;
a laryngeal cuff assembly coupled with the support assembly, wherein the laryngeal cuff assembly is positioned within the patient's supraglottic airway at least in part by the support assembly, wherein the laryngeal cuff assembly at least partially isolates and seals a portion of the supraglottic airway of the patient, and wherein the laryngeal cuff assembly operates to assist in positioning a distal section of the intrathoracic pressure monitoring lumen in fluid communication with the patient's trachea, during administration of the resuscitative procedure to the patient;
an esophageal cuff assembly coupled with the support assembly distal to the laryngeal cuff assembly, wherein the esophageal cuff assembly interfaces with the esophagus of the patient during administration of the resuscitative procedure to the patient; and
a fluid passage circuit defined at least in part by the esophageal cuff assembly and in fluid communication with the fluid delivery lumen and the fluid return lumen of the support assembly.

2. The airway adjunct resuscitation system according to claim 1, further comprising an intrathoracic pressure regulation mechanism in operative association with the support assembly, wherein the intrathoracic pressure regulation mechanism is adapted for use in regulating intrathoracic pressure within the patient.

3. The airway adjunct resuscitation system according to claim 2, wherein the intrathoracic pressure regulation mechanism comprises a member selected from the group consisting of an Impedance Threshold Device (ITD) mechanism and an Intrathoracic Pressure Regulator (ITPR) mechanism.

4. (canceled)

5. (canceled)

6. The airway adjunct resuscitation system according to claim 1, wherein the esophageal cuff assembly operates to provide a support against which the patient's heart is compressed during administration of the resuscitative procedure, so as to increase efficiency of the resuscitative procedure.

7. The airway adjunct resuscitation system according to claim 1, wherein the esophageal cuff assembly operates to provide a cooling treatment to the patient.

8. The airway adjunct resuscitation system according to claim 1, wherein the esophageal cuff assembly operates to (i) assist in stabilizing the airway adjunct resuscitation system within the patient, (ii) provide a support against which the patient's heart is compressed during administration of the resuscitative procedure so as to increase efficiency of the resuscitative procedure, and (iii) provide a cooling treatment to the patient.

9. (canceled)

10. (canceled)

11. (canceled)

12. The airway adjunct resuscitation system according to claim 1, wherein the support assembly defines an auxiliary lumen for use in monitoring a physiological parameter within the patient.

13. The airway adjunct resuscitation system according to claim 1, further comprising an intrathoracic pressure monitoring mechanism in fluid communication with the intrathoracic pressure monitoring lumen, wherein the intrathoracic pressure monitoring mechanism monitors pressure within the intrathoracic pressure monitoring lumen during administration of the resuscitative procedure to the patient.

14. (canceled)

15. (canceled)

16. (canceled)

17. (canceled)

18. (canceled)

19. The airway adjunct resuscitation system according to claim 1, wherein the esophageal cuff assembly defines an esophageal balloon and the gastric cuff assembly defines a gastric balloon that is separate or continuous from the esophageal balloon.

20. The airway adjunct resuscitation system according to claim 1, wherein the esophageal cuff assembly and the gastric cuff assembly are contiguous.

21. (canceled)

22. (canceled)

23. The airway adjunct resuscitation system according to claim 1, wherein the laryngeal cuff assembly comprises an elastomeric gel body that is shaped to interface with contours of the patient's airway.

24. (canceled)

25. (canceled)

26. (canceled)

27. (canceled)

28. (canceled)

29. (canceled)

30. The airway adjunct resuscitation system according to claim 1, wherein the laryngeal cuff assembly comprises a laryngeal cuff balloon and the esophageal cuff assembly defines an esophageal cuff balloon, and wherein the laryngeal cuff balloon is contiguous with the esophageal cuff balloon, such that pressure, volume, or both can be redistributed between the laryngeal cuff balloon and the esophageal cuff balloon when pressure is applied to or released from the patient's sternum during administration of the resuscitative procedure to the patient when the resuscitative procedure comprises administration of external chest compressions.

31. The airway adjunct resuscitation system according to claim 1, wherein the esophageal cuff assembly provides a platform against which the patient's heart may be compressed during administration of the resuscitative procedure to the patient when the resuscitative procedure comprises administration of external chest compressions.

32. The airway adjunct resuscitation system according to claim 1, wherein the esophageal cuff assembly comprises a stimulation assembly.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. The airway adjunct resuscitation system according to claim 1, wherein the esophageal cuff assembly comprises a heat conducting material.

46. The airway adjunct resuscitation system according to claim 1, wherein the esophageal cuff assembly comprises a non-compressible and non-expansible material that resists rupture during administration of the resuscitative procedure to the patient when the resuscitative procedure comprises administration of external chest compressions.

47. The airway adjunct resuscitation system according to claim 1, further comprising a physiological sensors.

48. The airway adjunct resuscitation system according to claim 1, further comprising a communication mechanism.

49. (canceled)

50. (canceled)

51. A method of administering a resuscitative procedure to a patient, the method comprising:
engaging an airway adjunct resuscitation system with the patient, wherein the airway adjunct resuscitation system comprises:
a support assembly defining an intrathoracic pressure monitoring lumen;
an intrathoracic pressure monitoring mechanism in fluid communication with the intrathoracic pressure monitoring lumen;
a laryngeal cuff assembly coupled with the support assembly;
an esophageal cuff assembly coupled with the support assembly distal to the laryngeal cuff assembly; and
a gastric cuff assembly coupled with the support assembly distal to the esophageal cuff assembly;
positioning the laryngeal cuff assembly within the patient's supraglottic airway;
positioning a distal section of the intrathoracic pressure monitoring lumen in fluid communication with the patient's trachea;
isolating and sealing a portion of the supraglottic airway of the patient with the laryngeal cuff assembly;
placing the esophageal cuff assembly at the esophagus of the patient;
placing the gastric cuff assembly at the stomach of the patient, and
monitoring pressure within the intrathoracic pressure monitoring lumen with the intrathoracic pressure monitoring mechanism, so that the patient's trachea is exposed to the monitored pressure within the intrathoracic pressure monitoring lumen.

52. (canceled)

53. (canceled)

54. (canceled)

55. (canceled)

56. The method according to claim 51, further comprising engaging a bite block mechanism of the airway adjunct resuscitation system with the patient's mouth.

57. (canceled)

58. The method according to claim 51, further comprising monitoring a physiological parameter with a physiological sensor of the airway adjunct resuscitation system.

59. (canceled)

60. (canceled)

61. (canceled)

62. (canceled)

63. The method according to claim 51, further comprising administering repeated chest compressions to the patient.

64. The method according to claim 51, wherein the intrathoracic pressure monitoring mechanism comprises a sequential valve system configured to administer no inspiratory resistance for a positive pressure ventilation, no expiratory resistance, an optional PEEP, or a preset or variable resistance to inflow of respiratory gases.

65. The method according to claim 51, wherein the intrathoracic pressure monitoring mechanism is configured to generate a continuous negative intrathoracic pressure with intermittent positive pressure ventilation.

66. The method according to claim 51, further comprising monitoring an end tidal CO₂ level in the patient with an end tidal CO₂ sensor of the airway adjunct resuscitation system, and adjusting the resuscitative procedure based on the end tidal CO₂ level.

67. A method for treating a patient suffering from or at risk of developing a condition selected from the group consisting of heart failure, cardiac arrest, sepsis, shock, acute respiratory distress syndrome, polytrauma, head disease, elevated hepatic or portal vein pressures, bleeding during abdominal, head and neck surgery, and insufficient circulation during open heart surgery, the method comprising:
engaging an airway adjunct resuscitation system with the patient, wherein the airway adjunct resuscitation system comprises:
a support assembly defining an intrathoracic pressure monitoring lumen;
an intrathoracic pressure monitoring mechanism in fluid communication with the intrathoracic pressure monitoring lumen;
a laryngeal cuff assembly coupled with the support assembly;
an esophageal cuff assembly coupled with the support assembly distal to the laryngeal cuff assembly; and
a gastric cuff assembly coupled with the support assembly distal to the esophageal cuff assembly;
positioning the laryngeal cuff assembly within the patient's supraglottic airway;
positioning a distal section of the intrathoracic pressure monitoring lumen in fluid communication with the patient's trachea;
isolating and sealing a portion of the supraglottic airway of the patient with the laryngeal cuff assembly;
placing the esophageal cuff assembly at the esophagus of the patient;
placing the gastric cuff assembly at the stomach of the patient; and
monitoring pressure within the intrathoracic pressure monitoring lumen with the intrathoracic pressure monitoring mechanism, so that the patient's trachea is exposed to the monitored pressure within the intrathoracic pressure monitoring lumen.

68. A method for treating a patient suffering from or at risk of developing low circulation, the method comprising:
engaging an airway adjunct resuscitation system with the patient, wherein the airway adjunct resuscitation system comprises:
a support assembly defining an intrathoracic pressure monitoring lumen;
an intrathoracic pressure monitoring mechanism in fluid communication with the intrathoracic pressure monitoring lumen;
a laryngeal cuff assembly coupled with the support assembly;
an esophageal cuff assembly coupled with the support assembly distal to the laryngeal cuff assembly; and
a gastric cuff assembly coupled with the support assembly distal to the esophageal cuff assembly;
positioning the laryngeal cuff assembly within the patient's supraglottic airway;
positioning a distal section of the intrathoracic pressure monitoring lumen in fluid communication with the patient's trachea;
isolating and sealing a portion of the supraglottic airway of the patient with the laryngeal cuff assembly;
placing the esophageal cuff assembly at the esophagus of the patient;
placing the gastric cuff assembly at the stomach of the patient; and
expanding the esophageal cuff assembly so as to anteriorly move the heart within the patient's body;
administering of external chest compressions to the patient so as to compress the patient's heart against the esophageal cuff assembly; and
monitoring pressure within the intrathoracic pressure monitoring lumen with the intrathoracic pressure monitoring mechanism, so that the patient's trachea is exposed to the monitored pressure within the intrathoracic pressure monitoring lumen.

69. A method for providing a cooling treatment to a patient, the method comprising:
engaging an airway adjunct resuscitation system with the patient, wherein the airway adjunct resuscitation system comprises:
a support assembly defining an intrathoracic pressure monitoring lumen;
an intrathoracic pressure monitoring mechanism in fluid communication with the intrathoracic pressure monitoring lumen;
a laryngeal cuff assembly coupled with the support assembly;
an esophageal cuff assembly coupled with the support assembly distal to the laryngeal cuff assembly; and
a gastric cuff assembly coupled with the support assembly distal to the esophageal cuff assembly;
positioning the laryngeal cuff assembly within the patient's supraglottic airway;
positioning a distal section of the intrathoracic pressure monitoring lumen in fluid communication with the patient's trachea;
isolating and sealing a portion of the supraglottic airway of the patient with the laryngeal cuff assembly;
placing the esophageal cuff assembly at the esophagus of the patient;
placing the gastric cuff assembly at the stomach of the patient; and
introducing a cooling fluid into a member selected from the group consisting of the esophageal cuff assembly and the gastric cuff assembly.
